(19) United States
(12) Patent Application Publication
(10) Pub. No.: US 2005/0288369 A1
(43) Pub. Date: Dec. 29, 2005

(54) Novel n-hydroxy thiourea, urea and amide compounds and the pharmaceutical compositions comprising the same

(76) Inventor: Jee-Woo Lee, Seoul (KR)

Correspondence Address: KIRK HAHN, 14431 HOLT AVE, SANTA ANA, CA, 92705, US

(21) Appl. No.: 10/531,684

(22) PCT Filed: Oct. 17, 2003
(86) PCT No.: PCT/KR03/02175

§ 371 Date: Apr. 16, 2005

(30) Foreign Application Priority Data
Oct. 17, 2002 (KR) 10 2002 0063414

(51) Int. Cl. A61K 31/24; A61K 31/19; C07C 259/04

(52) U.S. Cl. 514/534; 514/575; 560/41; 562/621

(57) ABSTRACT

The present invention relates to novel n-hydroxythiourea, urea and amide compounds as a potent vanilloid receptor antagonist and the pharmaceutical compositions comprising the same. The inventive compound can be useful for analgesics to prevent, alleviate or treat pain diseases or inflammatory disease comprising pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease, inflammatory disease and urgent urinary incontinence.

### TECHNICAL FIELD

The present invention relates to novel n-hydroxythiourea, urea and amide compounds as a potent vanilloid receptor antagonist and the pharmaceutical compositions comprising the same.

### BACKGROUND ART

Capsaicin (8-methyl-N-vanillyl-6-nonenamides; CAP) is a main pungent component in hot pepper. Hot pepper has been used, for a long time, not only as a spice but also as a traditional medicine in the treatment of gastric disorders and when applied locally, for the relief of pain and inflammation (Szallasi and Blumberg, Pharm. Rev., 51, pp 159-211, 1999). CAP has wide spectra of biological actions, and not only exhibits effects on the cardiovascular and respiratory systems but also induces pain and irritancy on local application. However, CAP after such induction of pain induces desensitization to both CAP itself and other noxious stimuli to make the pain stopped. Based on those properties, CAP and its analogues such as olvanil, nuvanil, DA-5018, SDZ-249482, resiniferatoxin have been either used as an analgesic agent, therapeutic agent for incontinentia urinae or skin disorder and under development (Wriggleworth and Walpore, Drugs of the Future, 23, pp 531-538, 1998).

Transmissions for the mechanical, thermal and chemical noxious stimuli mainly occurred by primary afferent nerve fibers of fine unmyelinated nerve (C-fiber) and thin myelinated nerve (A-fiber), and main reaction site of CAP and its analogues called as vanilloid is present at the nerve fiber transmitting the noxious stimuli. CAP acts on the receptor existing on those neurons to induce potent irritation caused by potent inflow of mono-valent and di-valent cations such as calcium or sodium ion, and then exhibits potent analgesic effect by blocking the nervous function (Wood et al.; J. Neurosci., 8, pp 3208-3220, 1988).

Vanilloid receptor-1 (VR-1) has been recently cloned and its existence becomes clear (Caterina et al.; Nature, 389, pp 816-824, 1997). It has been clarified that this receptor transmits not only the stimuli by CAP analogues (vanilloid) but also various noxious stimuli such as proton, thermal stimuli etc. (Tominaga et al.; Neuron, 21, pp 513-543, 1998). Based on this, it is considered that VR functions as an integrative modulator against various noxious stimuli and carries out critical role in the transmission of pain and noxious stimuli. Recently, knock-out mouse in which gene encoding for vanilloid receptor was deleted, was prepared (Caterinal et al.; Science, 288, pp 306-313, 2000: Davis et al.; Nature, 405, pp 183-187, 2000). Compared with normal mice, the knock-out mouse was found out to exhibit significantly reduced response to thermal stimuli and thermal pain, while no difference in the respect of general behavior, of which result reconfirms the importance of VR in the transmission of noxious sensor. However, other endogenous ligand excepting proton, not exogenous ligand such as CAP, has been not known to be actually involved in transmission of noxious stimuli at VR till now.

In accordance with the study of present inventors, it has been confirmed that leukotrienes metabolites such as 12-hydroperoxyeicosatetraenoic acids (Hwang et al., Proc. Natl. Acad. Sci. U.S.A., 11, pp 6155-6160, 2000) and arachidonic acid such as anandamide (Zygmunt et al., Trends in Pharmacol. Sci., 21, pp 43-44, 2000) act as an endogenous ligand on vanilloid receptor but proton is regarded as a receptor-activating cofactor rather than a direct ligand.

Capsaicin-reactive sensory neuron and the vanilloid receptor existing therein are distributed to the whole body and act on the expression of inflammation besides basic function such as the transmission of pain and noxious signal, which is related to asthma, anaphylactic urinary bladder hypersensitiveness, irritable bowel syndrome and the etiology of skin disease.

Nowadays, the role of afferent sensory nerve showing reactivity on capsaicin in gastrointestinal damage has been highlighted and it causes to release peripheral neuronal peptide such as calcitonin gene-related peptide in order to improve the micro blood flow in as well as to show the contradict property of the protecting gastric injury and inducing gastric injury by the stimulation of sympathetic nervous system Men et al., Dig. Dis. Sci., 45, pp 830-836, 2000). Vanilloid receptor antagonist blocking vanilloid receptor, can be used for the purpose of preventing or treating above-mentioned various diseases.

Through binding endogenous pain-inducing molecules such as anandamide or HETE to receptor, the cations are influxed into a neuron to transmit the pain.

Antagonists competently inhibit the pain-inducing molecules from binding to receptor so that they can be used as analgesics with no side effect, occurring in the treatment by using agonist thereof such as initial irritancy.

Capsazepine, capsazocaine and ruthenium complex have been known as vanilloid receptor antagonists. The antagonistic effect of capsazocaine has not been reported at the level of receptor and ruthenium red has been known as a noncompetitive antagonist. Therefore, capsazepine has been reported as only one among true receptor competitive antagonists, which been paid attention to for the development of analgesics

The present inventors have made extensive researches to discover novel analgesic agents based on the above studies and finally completed the invention by the synthesis of N-(4-sulfonylamido)benzyl thiourea derivative and (4-sulfonylamido)phenyl acetamide derivative compound having excellent solubility and analgesic activity from the thiourea compound disclosed in the Korea patent application No. 2001-50092 and No. 2001-50093, the disclosure of which cited documents are incorporated herein by reference.

### DISCLOSURE OF THE INVENTION

Thus, the present invention provides novel compounds represented by the following general formula (I), the pharmaceutically acceptable salt or the isomer thereof: 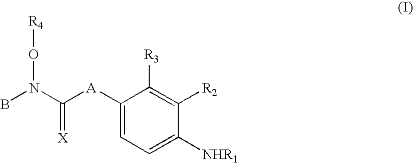
wherein
X is an oxygen or sulfur atom;
A is an aminomethylene or methylene group;
B is a 4-tert-butylbenzyl, a 3,4-dimethylphenylpropyl, an oleyl or 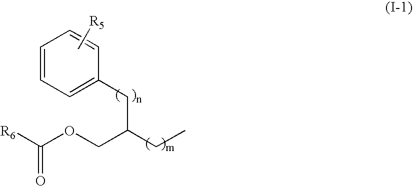
   group wherein m is integer of 0 or 1 and n is 1 or 2;
R₁ is a halogen-substituted or unsubstituted lower alkylsulfone having 1 to 5 carbon atoms, arylsulfone or a lower alkylcarbonyl group having 1 to 5 carbon atoms;
R₂ is a hydrogen atom, a methoxy group or halogen atom;
R₃ is a hydrogen atom, a methoxy group or halogen ato m;
R₄ is a hydrogen atom or a lower alkyl group having 1 to 5 carbon atoms;
R₅ is a hydrogen atom or a lower alkyl group having 1 to 5 carbon atoms;
R₆ is a lower alkyl group having 1 to 5 carbon atoms or a phenyl group.

It is another object of the present invention to provide the pharmaceutical composition comprising an efficient amount of the compound represented by general formula (I) or the pharmaceutically acceptable salt thereof as an active ingredient in amount effective to alleviate or treat pain diseases or inflammatory diseases together with pharmaceutically acceptable carriers or diluents.

The group having general formula (I) wherein R₁ is a methylsulfonyl group; R₂ is a hydrogen atom, a methoxyl group or a halogen atom; R₃ is a hydrogen atom or a halogen atom; R₄ is a hydrogen atom; X is an oxygen atom or a sulfur atom; A is an aminomethylene group; B is 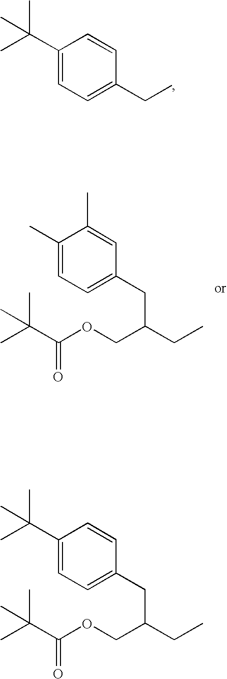
group are preferable.

Accordingly, the present invention also provides the compounds represented by following general formula (III), the pharmaceutically acceptable salt or the isomer thereof: 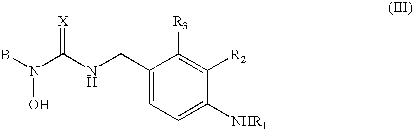
wherein the definitions of X, B, R₁, R₂ and R₃ substituents are same as those of general formula (I).

In preferred embodiment, the most preferred compound is one selected from the group consisting of;
N-(4-tert-butylbenzyl)-N-hydroxy-N-[4-(methylsulfonylamino)benzyl]thiourea,
N-(4-tert-butylbenzyl)-N-hydroxy-N-[3-methoxy-4-(methylsulfonylamino) benzyl]thiourea,
N-(4-tert-butylbenzyl)-N-hydroxy-N-[3-fluoro-4-(methylsulfonylamino)benzyl] thiourea,
N-(4-tert-butylbenzyl)-N-hydroxy-N-[3-chloro-4-(methylsulfonylamino)benzyl] thiourea,
N-(4-tert-butylbenzyl)-N-hydroxy-N-[4-(methylsulfonylamino)-3-nitrobenzyl] thiourea,
N-(4-tert-butylbenzyl)-N-hydroxy-N-[2-fluoro-4-(methylsulfonylamino)benzyl] thiourea,
N-(4-tert-butylbenzyl)-N-hydroxy-N-[2-chloro-4-(methylsulfonylamino)benzyl] thiourea,
N-[2-(3,4-dimethylbenzyl)-3-(pivaloyloxy)propyl]-N-hydroxy-N-[4-(methylsulfonylamino)benzyl] thiourea,
N-[2-(3,4-dimethylbenzyl)-3-(pivaloyloxy)propyl]-N-hydroxy-N-[3-methoxy-4-(methylsulfonylamino)benzyl] thiourea,
N-[2-(3,4-dimethylbenzyl)-3-(pivaloyloxy)propyl]-N-hydroxy-N-[3-fluoro-4-(methylsulfonylamino)benzyl] thiourea,
N-[2-(3,4-dimethylbenzyl)-3-(pivaloyloxy)propyl]-N-hydroxy-N-[2-fluoro-4-(methylsulfonylamino)benzyl] thiourea,
N-[2-(3,4-dimethylbenzyl)-3-(pivaloyloxy)propyl]-N-hydroxy-N-[2-chloro-4-(methylsulfonylamino)benzyl] thiourea,
N-[2-(4-tert-butylbenzyl)-3-(pivaloyloxy)propyl]-N-hydroxy-N-[4-(methylsulfonylamino)benzyl] thiourea,
N-[2-(4-tert-butylbenzyl)-3-(pivaloyloxy)propyl]-N-hydroxy-N-[3-fluoro-4-(methylsulfonylamino)benzyl] thiourea.

The group having general formula (I), wherein R₁ is a methylsulfonyl group; R₂ is a hydrogen atom, a methoxyl group or a halogen atom; R₃ is a hydrogen atom or a halogen atom; R₄ is a hydrogen atom; X is an oxygen atom; Y is a nitrogen atom; A is an methylene group; B is 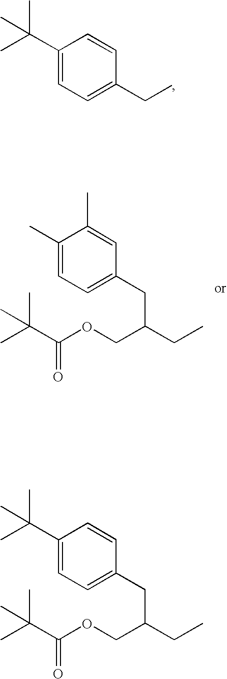
group are preferable.

Accordingly, present invention also provides the compound represented by general formula (IV), the pharmaceutically acceptable salt or the isomer thereof: 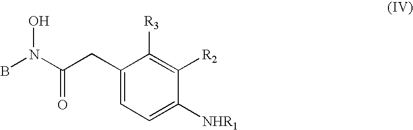
wherein the definitions of B, R₁, R₂ and R₃ substituents are same as those of general formula (I).

In preferred embodiment, the most preferred compound comprises N-(4-tert-butylbenzyl)-N-hydroxy-[4-(methylsulfonylamino)phenyl] acetamide.

Also, it is another object of the present invention to provide compound represented by general formula (II) or the pharmaceutically acceptable salt or the isomer thereof. 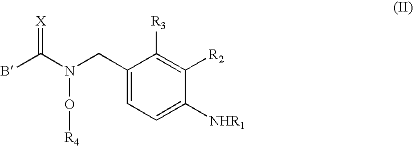
wherein
X is an oxygen or sulfur atom;
B′ is an aforementioned B or a secondary amine substituted with B;
R₁ is a halogen-substituted or unsubstituted lower alkylsulfone having 1 to 5 carbon atoms, arylsulfonyl group or lower alkylcarbonyl group having 1 to 5 carbon atoms;
R₂ is a hydrogen atom, a methoxy group or halogen atom;
R₃ is a hydrogen atom, a methoxy group or halogen atom;
R₄ is a hydrogen atom or a lower alkyl group having 1 to 5 carbon atoms.

It is another object of the present invention to provide the pharmaceutical composition comprising the compound having general formula (II) or the pharmaceutically acceptable salt thereof as an active ingredient in amount effective to alleviate or treat pain diseases or inflammatory diseases together with pharmaceutically acceptable carrier or diluents.

The group having general formula (II) wherein B′ is a secondary amine group substituted with aforementioned B; R₁ is a methylsulfonyl group; R₂ is a hydrogen atom or a halogen atom; R₃ is a hydrogen atom; R₄ is a hydrogen atom; X is an oxygen atom or a sulfur atom are preferable as the third group.

Accordingly, present invention also provides the compound represented by general formula (V), the pharmaceutically acceptable salt or the isomer thereof: 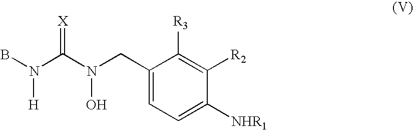
wherein the definitions of X, B, R₁, R₂ and R₃ substituents are same as those of general formula (I).

In preferred embodiment, the most preferred compound is one selected from the group consisting of
N-(4-tert-butylbenzyl)-N-hydroxy-N-[4-(methylsulfonylamino)benzyl]thiourea,
N-[2-(3,4-dimethylbenzyl)-3(pivaloyloxy)propyl]-N-hydroxy-N-[4-(methylsulfonyl amino)benzyl]thiourea,
N-(4-tert-butylbenzyl)-N-hydroxy-N-[4-(methylsulfonylamino)benzyl]urea,
N-[2-(3,4-dimethylbenzyl)-3(pivaloyloxy)propyl]-N-hydroxy-N-[3-fluoro-4-(methylsulfonylamino)benzyl]thiourea.

The group having general formula (II) wherein B′ is an aforementioned B; R₁ is a methylsulfonyl group; R₂ is a hydrogen atom, a methoxyl group or a halogen atom; R₃ is a hydrogen atom or a halogen atom; R is a hydrogen atom; X is an oxygen atom are preferable as the fourth group.

The present invention also provides the compound represented by general formula (VI), the pharmaceutically acceptable salt or the isomer thereof: 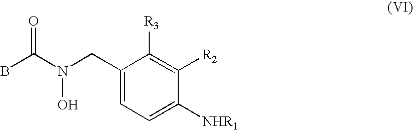
wherein the definitions of B, R₁, R₂ and R₃ substituents are same as those of general formula (I).

The preferred compound comprises N-hydroxy-N-[4-(methylsulfonylamino)benzyl]-2-(4-tert-butylphenyl)acetamide.

The inventive compounds represented by general formula (I) or (II) can be transformed into their pharmaceutically acceptable salt and solvates by the conventional method well known in the art. For the salts, acid-addition salt thereof formed by a pharmaceutically acceptable free acid thereof is useful and can be prepared by the conventional method. For example, after dissolving the compound in the excess amount of acid solution, the salts are precipitated by the water-miscible organic solvent such as methanol, ethanol, acetone or acetonitrile to prepare acid addition salt thereof and further the mixture of equivalent amount of compound and diluted acid with water or alcohol such as glycol monomethylether, can be heated and subsequently dried by evaporation or filtrated under reduced pressure to obtain dried salt form thereof.

As a free acid of above-described method, organic acid or inorganic acid can be used. For example, organic acid such as methansulfonic acid, p-toluensulfonic acid, acetic acid, trifluoroacetic acid, citric acid, maleic acid, succinic acid, oxalic acid, benzoic acid, lactic acid, glycolic acid, gluconic acid, galacturonic acid, glutamic acid, glutaric acid, glucuronic acid, aspartic acid, ascorbic acid, carbonylic acid, vanillic acid, hydroiodic acid and the like, and inorganic acid such as hydrochloric acid, phosphoric acid, sulfuric acid, nitric acid, tartaric acid and the like can be used herein.

Further, the pharmaceutically acceptable metal salt form of inventive compounds may be prepared by using base. The alkali metal or alkali-earth metal salt thereof can be prepared by the conventional method, for example, after dissolving the compound in the excess amount of alkali metal hydroxide or alkali-earth metal hydroxide solution, the insoluble salts are filtered and remaining filtrate is subjected to evaporation and drying to obtain the metal salt thereof. As a metal salt of the present invention, sodium, potassium or calcium salt are pharmaceutically suitable and the corresponding silver salt can be prepared by reacting alkali metal salt or alkali-earth metal salt with suitable silver salt such as silver nitrate.

The pharmaceutically acceptable salt of the compound represented by general formula (I) or (II) comprise all the acidic or basic salt which may be present at the compounds, if it does not indicated specifically herein. For example, the pharmaceutically acceptable salt of the present invention comprise the salt of hydroxyl group such as the sodium, calcium and potassium salt thereof; the salt of amino group such as the hydrogen bromide salt, sulfuric acid salt, hydrogen sulfuric acid salt, phosphate salt, hydrogen phosphate salt, dihydrophosphate salt, acetate salt, succinate salt, citrate salt, tartarate salt, lactate salt, mandelate salt, methanesulfonate(mesylate) salt and p-toluenesulfonate (tosylate) salt etc, which can be prepared by the conventional method well known in the art.

There may exist in the form of optically different diastereomers since the compounds represented by general formula (I) or (II) have unsymmetrical centers, accordingly, the compounds of the present invention comprise all the optically active isomers, R or S stereoisomers and the mixtures thereof. Present invention also comprises all the uses of racemic mixture, more than one optically active isomer or the mixtures thereof as well as all the preparation or isolation method of the diastereomer well known in the art.

The compounds of the invention of formula (I) or (II) may be chemically synthesized by the methods which will be explained by following reaction schemes hereinafter, which are merely exemplary and in no way limit the invention. The reaction schemes show the steps for preparing the representative compounds of the present invention, and the other compounds also may be produced by following the steps with appropriate modifications of reagents and starting materials, which are envisaged by those skilled in the art.

### General Synthetic Procedures

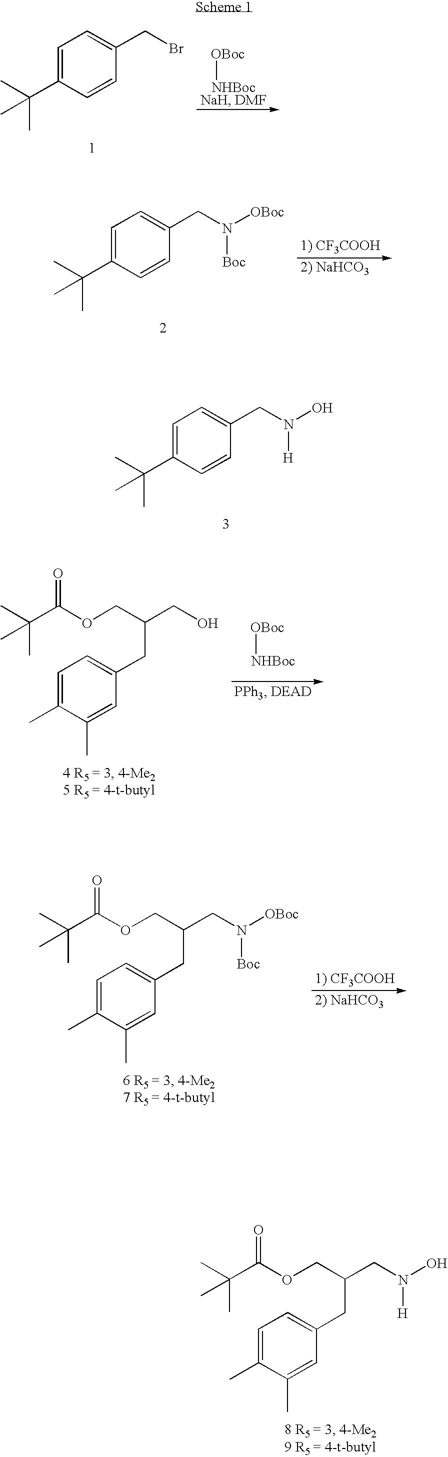

As depicted in above Scheme 1,4-tert-buthylbenzyl bromide 1 is reacted with tert-butyl-N-(tert-butoxycarbonyloxy)carbamate under the basic condition to synthesize compound 2, and then Boc(tert-butoxycarbonyl) group of compound 2 is removed under the acidic condition to synthesize hydroxylamine compound 3.

Compound 4 or 5 is condensed with tert-butyl-N-(tert-butoxycarbonyloxy) carbamate according to Mitsunobu reaction to synthesize compound 6 or 7 and subsequently hydroxylamine compound 8 and 9 are synthesized by removing deprotection group of compound 6 or 7.

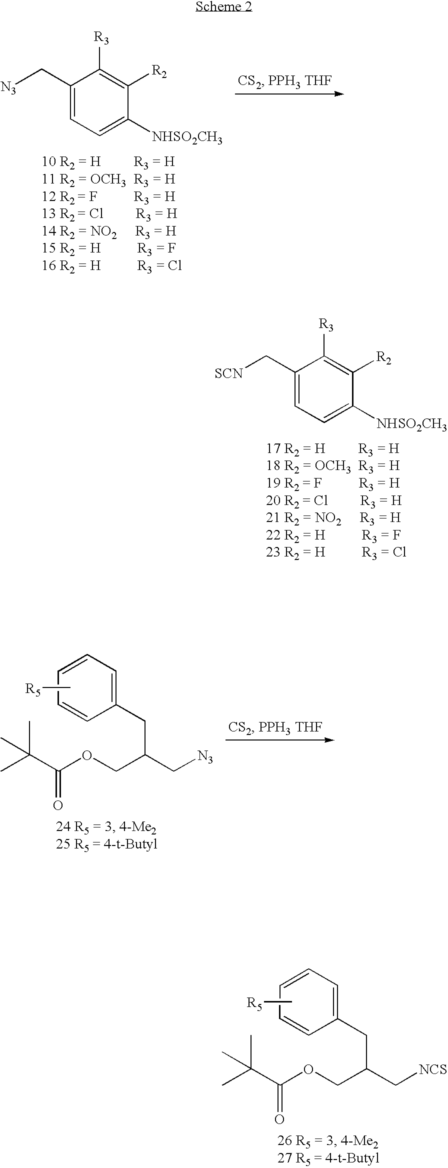

As depicted in the above Scheme 2, the azide compounds 10 to 16 and 24, 25 disclosed in Korea patent application Nos. 2001-50092 and 2001-50093 are reacted with PPh₃ and CS₂ to produce isothiocyanate compound 17 to 23, 26 and 27.

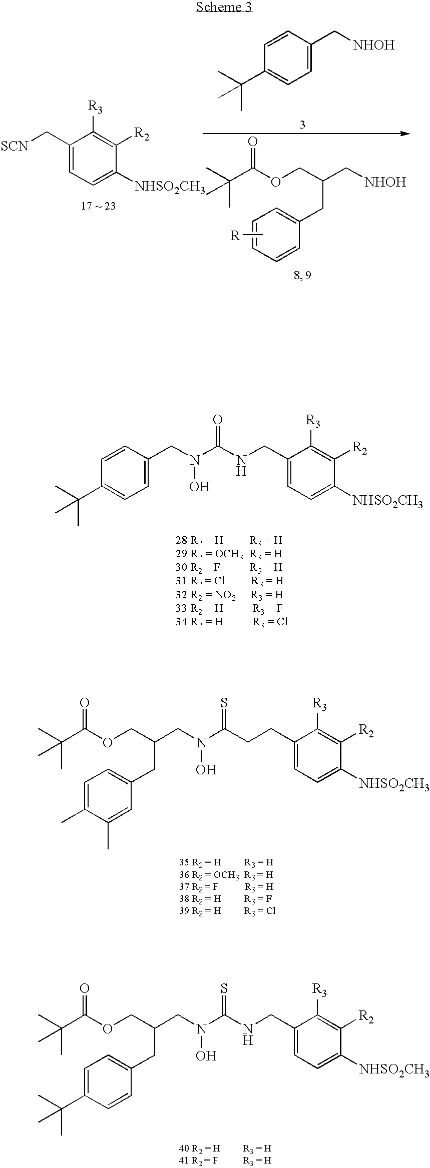

As depicted in the above Scheme 3, the isothiocyanate compound 17 to 23 of scheme 2 is condensed with hydroxylamine 3 and compound 8 or 9 to synthesize N-hydroxy thiourea compounds 28 to 41 having methylsulfonylaminobenzyl group.

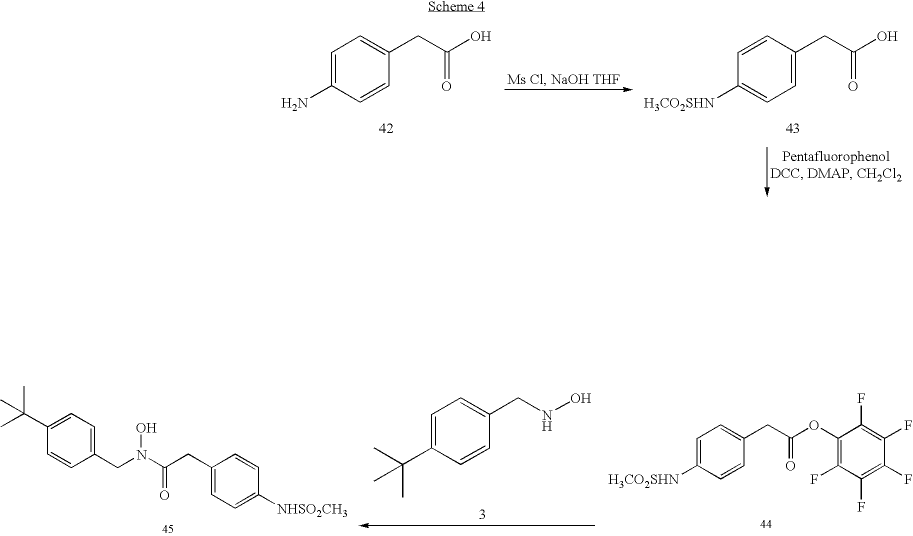

As shown in the above Scheme 4,4-aminophenylacetic acid 42 is used as a starting material, and its amine group is mesylated and its acid moiety is converted to pentafluorophenylester to produce compound 44.

The compound 44 is condensed with hydroxylamine 3 to synthesize N-hydroxy amide compound 45 having 4-methylsulfonylaminobenzyl group.

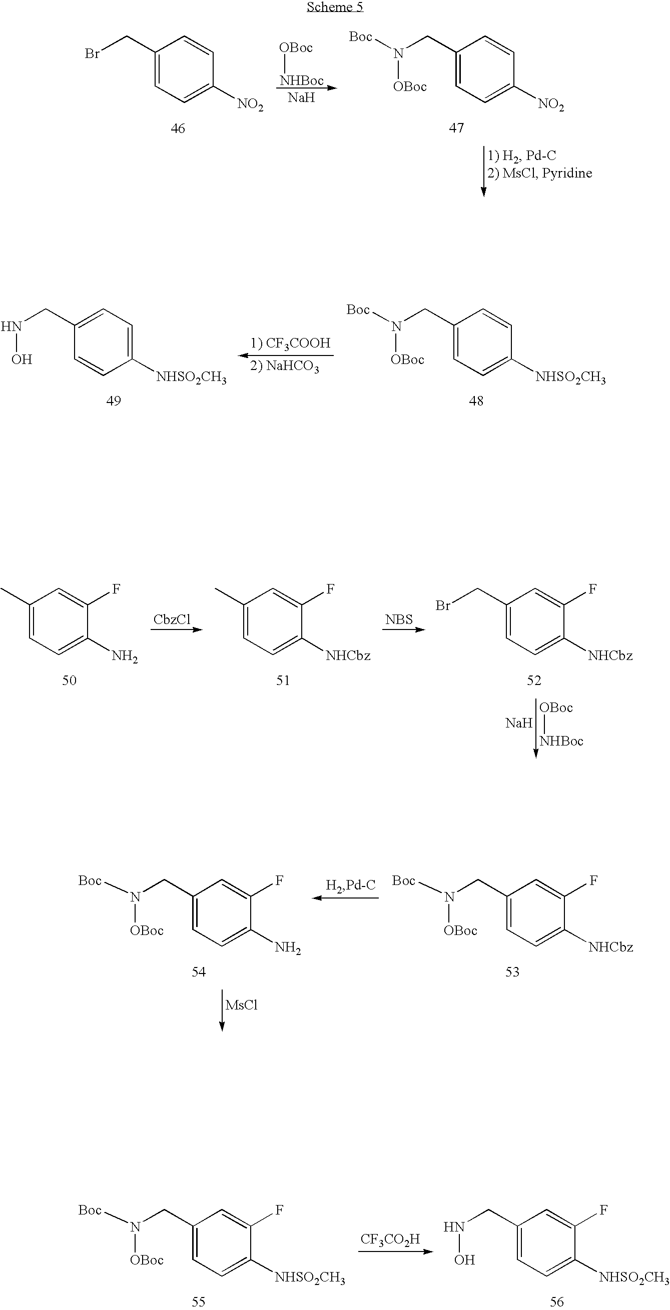

As shown in Scheme 5, 4-nitrobenzyl bromide 46 as a starting material is reacted with tert-butyl-N-(tert-butoxylcarbonyloxy)carbamate under the basic condition to synthesize compound 47 and after reducing the nitro group thereof, the mesylation is performed to synthesize the compound 48. And then the Boc protecting group is removed under acidic condition with sodium bicarbonate to produce hydroxylamine compound 49.

In the synthesis of 3-fluoro derivative of compound 49, the amine group of 2-fluoro-4-methylaniline 50 as a starting material is protected with carbobenzoxy group (Cbz) and the methyl group thereof is brominated to synthesize compound 52. The compound 52 is reacted with tert-butyl-N-(tert-butoxycarbonyloxy)carbamate under basic condition to produce compound 53. After Cbz group of compound 53 is removed under catalytically reduction condition to produce compound 54 and the methanesulfone group thereof is condensed to synthesize compound 55. Finally, the Boc group is removed under acidic condition to obtain hydroxylamine compound 56.

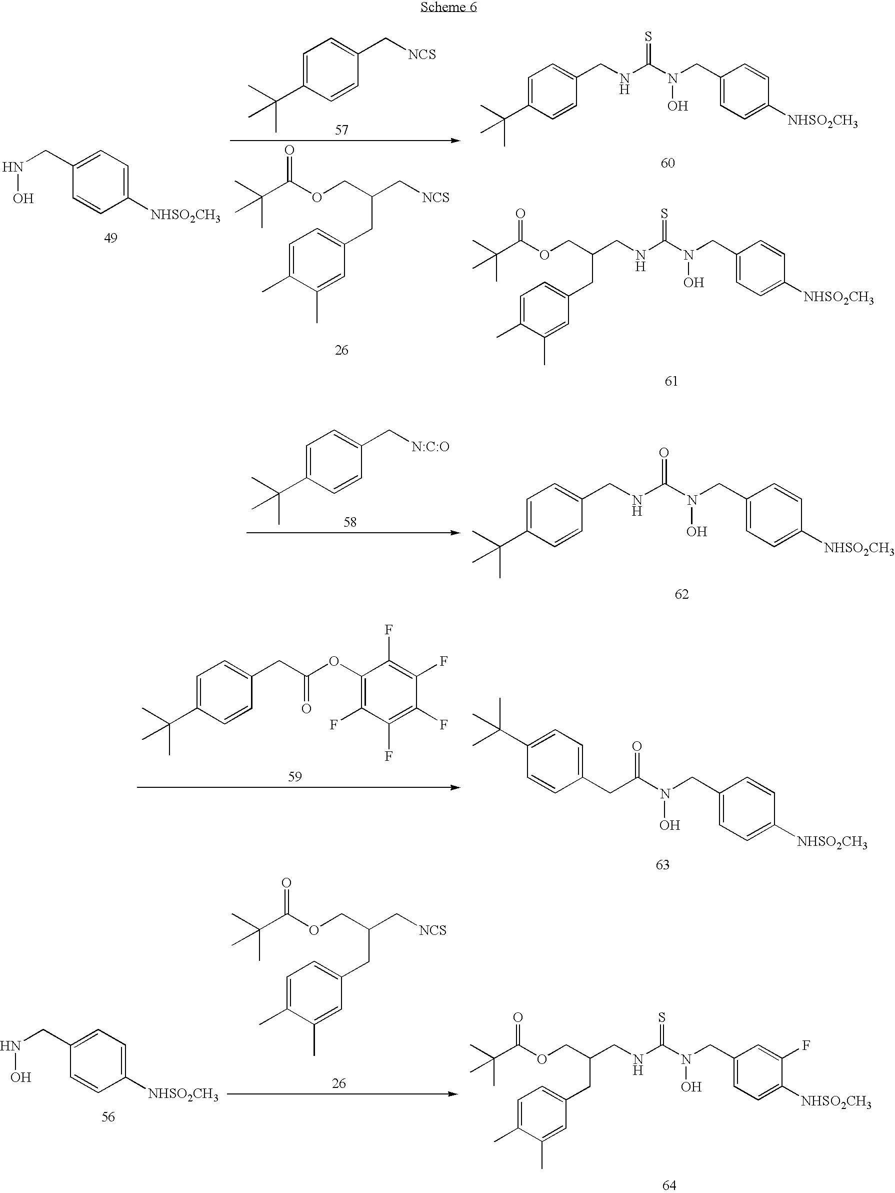

As shown in Scheme 6, hydroxylamine compound 49 is reacted with isothiocyanate 57 or compound 26 to synthesize N-hydroxythiourea compound 60 or 61, with isothianate 58 to synthesize N-hydroxythiourea compound 70 and with pentafluorophenylester 59 to synthesize compound 63, respectively.

Also, hydroxylamine compound 56 having 3-F group is condensed with isothiocyanate 26 to produce N-glemhydroxythiourea compound 64.

The present invention also provides a pharmaceutical composition comprising a compound of formula (I) or (II) or a pharmaceutically acceptable salt thereof as an active ingredient for an antagonist of vanilloid receptor.

The compound of formula (I) or (II) according to the present invention has potent analgesic and anti-inflammatory activity, and the pharmaceutical composition of the present invention thus may be employed to alleviate or relieve acute, chronic or inflammatory pains or to suppress inflammation and to treat urgent urinary incontinence.

The present invention also provides a pharmaceutical composition comprising the compound selected from the group consisting of compounds of formula (I) or (II) or the pharmaceutical acceptable salts thereof for preventing and treating pain diseases or inflammatory diseases.

Pain diseases or inflammatory diseases comprise at least one selected from the group consisting of pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease and the like.

The present invention also provides a pharmaceutical composition comprising the compound selected from the group consisting of compounds of formula (I) or (II) or the pharmaceutical acceptable salts thereof for preventing and treating urgent urinary incontinence.

The pharmaceutical composition of the present invention comprises the inventive compounds between 0.0001 to 10% by weight, preferably 0.0001 to 1% by weight based on the total weight of the composition.

The present invention also provides an use of compound selected from the group consisting of compounds of formula (I) or (II) or the pharmaceutical acceptable salts thereof as antagonists of vanilloid receptors.

In accordance with another aspect of the present invention, there is also provided an use of the compound (I) or (II) for manufacture of medicines employed for alleviating or treating pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease, inflammatory disease or urgent urinary incontinence.

The compound of formula (I) or (II) according to the present invention can be provided as a pharmaceutical composition comprising pharmaceutically acceptable carriers, adjuvants or diluents. For example, the compounds of the present invention can be dissolved in oils, propylene glycol or other solvents, which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

In accordance with another aspect of the present invention, there is also provided an method of alleviating or treating pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease, inflammatory disease or urgent urinary incontinence, wherein the method comprises administering a therapeutically effective amount of the compound of formula of (I) or (II) or the pharmaceutically acceptable salt thereof.

Hereinafter, the following formulation methods and excipients are merely exemplary and in no way limit the invention.

The compounds of the present invention in pharmaceutical dosage forms may be used in the form of their pharmaceutically acceptable salts, and also may be used alone or in appropriate association, as well as in combination with other pharmaceutically active compounds.

The compounds of the present invention may be formulated into preparations for injections by dissolving, suspending, or emulsifying them in aqueous solvents such as normal saline, 5% Dextrose, or non-aqueous solvent such as vegetable oil, synthetic aliphatic acid glycerides, esters of higher aliphatic acids or propylene glycol. The formulation may include conventional additives such as solubilizers, isotonic agents, suspending agents, emulsifying agents, stabilizers and preservatives.

The desirable dose of the inventive compounds varies depending on the condition and the weight of the subject, severity, drug form, route and period of administration, and may be chosen by those skilled in the art. However, in order to obtain desirable effects, it is generally recommended to administer at the amount ranging 0.0001-100 mg/kg, preferably 0.001-100 mg/kg by weight/day of the inventive compounds of the present invention. The dose may be administered in single or divided into several times per day. In terms of composition, the compounds should be present between 0.0001 to 10% by weight, preferably 0.0001 to 1% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intrathecal, epidural or intracerebroventricular injection.

It is anther object of the present invention to provide a use of the above-mentioned compound of the present invention for the preparation of therapeutic agent for the preventing and treating pain disease or inflammatory disease by showing vanilloid receptor-antagonistic activity in human or mammal.

Additionally, it is an object of the present invention to provide a method of treating or preventing pain disease and inflammatory disease by showing vanilloid receptor-antagonistic activity in a mammal comprising administering to said mammal an effective amount of the above-mentioned compound of the present invention together with a pharmaceutically acceptable carrier thereof.

It will be apparent to those skilled in the art that various modifications and variations can be made in the compositions, use and preparations of the present invention without departing from the spirit or scope of the invention.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows the analgesic effect of thiourea compounds in prior art (JYL-827, JYL-1433) and N-hydroxy thiourea compound 35 (SU-66) and 37 (SU-154) in acetic acid-induced writhing test.

### BEST MODE FOR CARRYING OUT THE INVENTION

The present invention is more specifically explained by the following examples. However, it should be understood that the present invention is not limited to these examples in any manner.

### EXAMPLE 1

### Preparation of tert-butyl-N-[(tert-butoxycarbonyl)oxy]-N-(4-tert-butylbenzyl)carbamate Compound (2)

A cooled solution of tert-butyl-N-(tert-butoxycarbonyloxy)carbamate (5 g, 21.4 mmol) in DMF (20 ml) at 0° C. was treated with sodium hydride (60%, 12.8 g, 21.4 mmol) portionwisely and stirred for 30 min at room temperature. The reaction mixture was added to 4-tert-butylbenzyl bromide (7.3 g, 32.1 mmol) and stirred for 18 hrs at room temperature. The mixture was diluted with H₂O and extracted with EtOAc several times. The combined organic layers were washed with H₂O and brine, dried over MgSO₄ and concentrated in vacuo. The residue was purified by column chromatography on Silica gel with EtOAc/hexanes (10:1) solvent mixture as an eluant to give 7.72 g of colorless tert-butyl-N-[(tert-butoxycarbonyl)oxy]-N-(4-tert-butylbenzyl) carbamate 2 (yield: 95%).

¹H-NMR (CDCl₃) δ: 7.35 (dt, 2H, J=2.2, 8.5 Hz, Ar), 7.26 (d, 2H, J=8.5 Hz, Ar), 4.72 (s, 2H, CH₂), 1.49 (s, 9H, C(CH₃)₃), 1.44 (s, 9H, C(CH₃)₃), 1.30 (s, 9H, C(CH₃)₃).

### EXAMPLE 2

### Preparation of N-[4-tert-butylbenzyl]hydroxylamine Compound (3)

A cooled solution of tert-butyl-N-[(tert-butoxycarbonyl)oxy]-N-(4-tert-butylbenzyl)carbamate of Example 1 (7.6 g, 20 mmol) in CH₂Cl₂ (100 ml) at 0° C. was treated with trifluoroacetic acid (20 ml) and stirred for 50 mins at room temperature. The mixture was concentrated in vacuo below 20° C. to remove the solvent. The residue was fractionated with saturated sodium bicarbonate and diethyl ester solution and the water soluble layer thereof was extracted with diethyl ester solution. The organic layers were washed with water and saline, dried over MgSO₄ and concentrated in vacuo to give 3.58 g of yellow oil of N-[4-tert-butylbenzyl]hydroxylamine 3 (yield: 100%).

¹H-NMR (CDCl₃) δ: 7.39 (d, 2H, J=8.0 Hz, Ar), 7.27 (d, 2H, J=8.0 Hz, Ar), 4.22 (s, 2H, CH₂), 1.27 (s, 9H, C(CH₃)₃).

### EXAMPLE 3

### Preparation of tert-butyl-N-[(tert-butoxycarbonyl)oxy]-N-[2-(3,4-dimethylbenzyl)-3-pivaloyloxy-propyl] carbamate Compound (6)

A solution of tert-butyl N-(tert-butoxycarbonyloxy)carbamate (0.92 g, 3.95 mmol) in THF (30 ml) was mixed with diethyl azodicarboxylate (0.85 ml, 5.39 mmol) slowly and stirred for 5 mins at room temperature. The mixture was reacted by the dropwise addition of triphenylphospine (1.41 g, 5.39 mmol) and above-mentioned compound 4 (1 g, 3.59 mmol) and stirred for 30 mins at room temperature. The reaction was stopped by adding 5 ml of methanol and the mixture was concentrated under reduced pressure. The residue was purified by column chromatography on Silica gel with EtOAc/hexanes (1:10) solvent mixture as an eluant to give 1.6 g of colorless oil of tert-butyl N-[(tert-butoxylcarbonyl)oxy]-N-[2-(3,4-dimethylbenzyl)-3-pivaloyloxy-propyl]carbamate compound 6 (yield: 90%).

¹H-NMR (CDCl₃) δ: 6.85-7.05 (m, 3H, Ar), 3.9-4.1 (m, 2H, CH₂OCO), 3.67 (bs, 2 H, CH₂N), 2.5-2.9 (m, 2H, CH₂A), 2.18-2.28 (m, 7H, 2×CH_{3 & CH),} 1.53 (s, 9H, C(CH₃)₃), 1.47 (s, 9H, C(CH₃)₃), 1.22 (s, 9H, C(CH₃)₃)

### EXAMPLE 4

### Preparation of tert-butyl-N-[(tert-butoxycarbonyl)oxy]-N-[2-(4-tert-butylbenzyl)-3-pivaloyloxy-propyl] carbamate Compound (7)

The compound 7 was prepared by the same procedure described in above Example 3 excepting using compound 5 to give 1.45 g of tert-butyl-N-[(tert-butoxycarbonyl)oxy]-N-[2-(4-tert-butylbenzyl)-3-pivaloyloxy-propyl] carbamate 7 (yield: 90%).

¹H-NMR (CDCl₃) δ: 7.29 (d, 2H, J=8.3 Hz, Ar), 7.09 (d, 2H, J=8.3 Hz, Ar), 4.00 (ddd of AB, 2H, CH₂OCO), 3.66 (bs, 2H, CH₂N), 2.79 (dd, 1H, CH₂A), 2.60 (dd, 1 H, CH₂A), 2.30 (m, 1H, CH), 1.52 (s, 9H, C(CH₃)₃), 1.47 (s, 9H, C(CH₃)₃), 1.30 (s, 9H, C(CH₃)₃), 1.22 (s, 9H, C(CH₃)₃)

### EXAMPLE 5

### Preparation of N-[2-(3,4-dimethylbenzyl)-3-pivaloyloxy-propyl] hydroxylamine Compound (8)

The compound 8 was prepared by the same procedure described in above Example 2 excepting using compound tert-butyl-N-[(tert-butoxycarbonyl)oxy]-N-[2-(3,4-dimethylbenzyl)-3-pivaloyloxy-propyl]carbamate compound 6 to give 1.6 g of N-[2-(3,4-dimethylbenzyl)-3-pivaloyloxy-propyl] hydroxylamine 8 (yield: 90%).

¹H-NMR(CDCl₃) δ: 6.86-7.06 (m, 3H, Ar), 5.45 (bs, 1H), 3.95-4.15 (m, 2H, CH₂OCO), 2.85-3.02 (m, 2H, CH₂N), 2.72 (d, 1H, CH₂A), 2.62 (m, 1H, CH₂A), 2.2-2.4 (m, 7H, 2×CH_{3 & CH)}

### EXAMPLE 6

### Preparation of N-[2-(4-tert-butylbenzyl)-3-pivaloyloxy-propyl] hydroxylamine Compound (9)

The compound 9 was prepared by the same procedure described in above Example 2 excepting using compound tert-butyl-N-[(tert-butoxycarbonyl)oxy]-N-[2-(4-tert-butylbenzyl)-3-pivaloyloxy-propyl]carbamate compound 7 to give 1.45 g of N-[2-(4-butylbenzyl)-3-pivaloyloxy-propyl] hydroxylamine 9 (yield: 88%).

¹H-NMR (CDCl₃) δ: 7.30 (d, 2H, J=8.2 Hz), 7.10 (d, 2H, J=8.2 Hz), 5.16 (bs, 1H), 4.06 (ddd of AB, 2H, J=5, 11.2 Hz, CH₂OCO), 2.95 (ddd of AB, 2H, J=6, 13 Hz, CH₂N), 2.67 (ddd of AB, 2H, J=7, 13.5 Hz, CH₂A), 2.33 (m, 1H, CH), 2.2-2.4 (m, 7H, 2×CH₃), 1.30 (s, 9H, C(CH₃)₃), 1.22 (s, 9H, C(CH₃)₃)

### EXAMPLE 7

### General Method of Isothiocyanate Synthesis

A mixture of azide (1.0 mmol), triphenylphosphine (290 mg, 1.1 mmol) in THF (10 ml) was treated with sodium hydride (NaH) (0.6 ml, 10 mmol), refluxed for 1 to 3 hours and concentrated in vacuo. The residue was purified by column chromatography with EtOAc/hexanes (1:2) solvent mixture as an eluant to give isothiocyanate compound.

### EXAMPLE 8

### Preparation of 4-(methylsulfonylamino)benzyl isothiocyanate Compound (17)

The white solid 4-(methylsulfonylamino)benzyl isothiocyanate compound 17 (yield: 63%) was prepared by the same procedure described in above Example 7.
melting point: 122-124° C.

¹H-NMR(CDCl₃) δ: 7.32 (d, 2H, J=8.4 Hz) 7.24 (d, 2H, J=8.4 Hz), 6.62 (s, 1H, NHSO₂), 4.70 (s, 2H, CH₂) 3.04 (s, 3H, SO₂CH₃)

### EXAMPLE 9

### Preparation of 3-methoxy-4-(methylsulfonylamino)benzyl isothiocyanate Compound (18)

The 3-methoxy-4-(methylsulfonylamino)benzyl isothiocyanate compound 18 (yield: 59%) was prepared by the same procedure described in above Example 7.
melting point: 100-103° C.

¹H-NMR(CDCl₃) δ: 7.53 (d, 1H, J=8.2 Hz), 6.88-6.92 (m, 2H), 6.80 (bs, 1H, NHSO₂), 4.68 (s, 2H, CH₂), 3.92 (s, 3H, OCH₃), 2.97 (s, 3H, SO₂CH₃)

### EXAMPLE 10

### Preparation of 3-fluoro-4-(methylsulfonylamino)benzyl isothiocyanate Compound (19)

The 3-fluoro-4-(methylsulfonylamino)benzyl isothiocyanate compound 19 (yield 54%) was prepared by the same procedure described in above Example 7.
melting point: 95-97° C.

¹H-NMR(CDCl₃) δ: 7.61 (t, 1H, J=8.0 Hz), 7.14 (m, 2H), 6.53 (bs, 1H, NHSO₂), 4.70 (s, 2H, CH₂), 3.01 (s, 3H, SO₂CH₃)

### EXAMPLE 11

### Preparation of 3-chloro-4-(methylsulfonylamino)benzyl isothiocyanate Compound (20)

The 3-chloro-4-(methylsulfonylamino)benzyl isothiocyanate compound 20 (yield 48%) was prepared by the same procedure described in above Example 7.
melting point: 112-113° C.

¹H-NMR(CDCl₃) δ: 7.68 (d, 1H, J=8.3 Hz), 7.42 (d, 1H, J=2.4 Hz), 7.26 (dd, 1H, J=8.3, 2.4 Hz), 6.80 (bs, 1H, NHSO₂), 4.70 (s, 2H, CH₂), 3.04 (s, 3H, SO₂CH₃)

### EXAMPLE 12

### Preparation of 4-(methylsulfonylamino)-3-nitrobenzyl isothiocyanate Compound (21)

The 4-(methylsulfonylamino)-3-nitrobenzyl isothiocyanate compound 21 (yield 42%) was prepared by the same procedure described in above Example 7.
melting point: 128-130° C.

¹H-NMR(CDCl₃) δ: 8.24 (d, 1H, J=2.4 Hz), 7.95 (d, 1H, J=8.3 Hz), 7.66 (dd, 1H, J=8.3, 2.4 Hz), 4.78 (s, 2H, CH₂), 3.18 (s, 3H, SO₂CH₃)

### EXAMPLE 13

### Preparation of 2-fluoro-4-(methylsulfonylamino)benzyl isothiocyanate Compound (22)

The 2-fluoro-4-(methylsulfonylamino)benzyl isothiocyanate compound 22 (yield 56%) was prepared by the same procedure described in above Example 7.

¹H-NMR(CDCl₃) δ: 7.38 (t, 1H, J=8.0 Hz), 7.09 (dd, 1H, J=10.9, 2.2 Hz), 6.99 (dd, 1H, J=8.3, 2.2 Hz), 4.73 (s, 2H, CH₂), 3.08 (s, 3H, SO₂CH₃)

### EXAMPLE 14

### Preparation of 2-chloro-4-(methylsulfonylamino)benzyl isothiocyanate Compound (23)

The 2-fluoro-4-(methylsulfonylamino)benzyl isothiocyanate compound 23 (yield 54%) was prepared by the same procedure described in above Example 7.
melting point: 110-112° C.

¹H-NMR (CDCl₃) δ: 7.43 (d, 1H, J=8.3 Hz), 7.33 (d, 1H, J=2.2 Hz), 7.16 (dd, 1 H, J=8.3 and 2.2 Hz), 6.79 (bs, 1H, NHSO₂), 4.79 (s, 2H, CH₂), 3.08 (s, 3H, SO₂CH₃)

### EXAMPLE 15

### Preparation of 2-(3,4-dimethylbenzyl)-3-pivaloyloxy-propyl isothiocyanate Compound (26)

The colorless oil of 2-(3,4-dimethylbenzyl)-3-pivaloyloxy propyl isothiocyanate compound 26 (yield: 92%) was prepared by the same procedure described in above Example 7.

¹H-NMR(CDCl₃) δ: 6.85-7.1 (m, 3H, Ar), 3.95-4.2 (m, 2H, CH₂OCO), 3.53 (m, 2H, CH₂NCS), 2.55-2.85 (m, 2H, CH₂A), 2.2-2.3 (m, 7H, 2×CH₃ and CH), 1.23 (s, 9H, C(CH₃)₃)

### EXAMPLE 16

### Preparation of 2-(4-tert-butylbenzyl)-3-pivaloyloxy-propyl isothiocyanate Compound (27)

The colorless oil of 2-(4-tert-butylbenzyl)-3-pivaloyloxy-propyl isothiocyanate compound 27 (yield: 90%) was prepared by the same procedure described in above Example 7.

¹H-NMR(CDCl₃) δ: 7.33 (d, 2H, J=8.3 Hz), 7.10 (d, 2H, J=8.3 Hz), 4.15 (dd, 1H, J=4.9, 11.4 Hz, CH₂OCO), 4.01 (dd, 1H, J=7, 11.4 Hz, CH₂OCO), 3.53 (sevenlet, 2H, CH₂NCS), 2.70 (ddd of AB, 2H, CH₂A), 2.31 (bs, 1H, CH), 1.31 (s, 9H, C(CH₃)₃), 1.23 (s, 9H, C(CH₃)₃).

### EXAMPLE 17

### General Method of N-hydroxythiourea Compound Synthesis

A mixture of hydroxylamine (1.0 mmol), isothiocyanate (1.0 mmol) in CH₂Cl₂ (10 ml) was stirred for 1 to 4 hours at room temperature and concentrated in vacuo. The residue was purified by column chromatography with EtOAc/hexanes (1:2) solvent mixture as an eluant to give N-hydroxythiourea compound.

### EXAMPLE 18

### Preparation of N-(4-tert-butylbenzyl)-N-hydroxy-N-[4-(methylsulfonylamino)benzyl]thiourea Compound (28)

The mixture of compound 17 and 3 was treated according to the same procedure described in above Example 17 to give white solid of N-(4-tert-butylbenzyl)-N-hydroxy-N-[4-(methylsulfonylamino)benzyl]thiourea compound 28 (yield: 94%).
melting point: 137° C.

¹H-NMR(CDCl₃) δ: 7.38 (s, 4H), 7.32 (d, 2H, J=8.3 Hz), 7.15 (d, 2H, J=8.3 Hz), 6.46 (s, 1H, NHSO₂), 5.97 (bs, 1H, NHCS), 5.34 (s, 2H, CH₂NOH), 4.82 (d, 2H, J=5.6 Hz, NHCH₂), 2.97 (s, 3H, SO₂CH₃), 1.31 (s, 9H, C(CH₃)₃) IR (KBr): 3350, 2962, 1512, 1336, 1123 cm⁻¹ MS m/z: 422 (MH⁺)

### EXAMPLE 19

### Preparation of N-(4-tert-butylbenzyl)-N-hydroxy-N-[3-methoxy-4-(methylsulfonylamino)benzyl]thiourea Compound (29)

The mixture of compound 18 and 3 was treated according to the same procedure described in above Example 17 to give white solid of N-(4-tert-butylbenzyl)-N-hydroxy-N-[3-methoxy-4-(methylsulfonylamino)benzyl]thiourea compound 29 (yield 92%) (See Table 1).
melting point: 112.5-115° C.

¹H-NMR (CDCl₃) δ: 7.39 (m, 4H), 6.99 (m, 1H), 6.91 (m, 1H), 6.74 (m, 1H), 5.52 (bs, 1H, NH), 5.36 (s, 2H, CH₂NHOH), 4.83 (d, 2H, J=5.6 Hz, CH₂NH), 3.88 (s, 3 H, OCH₃), 2.94 (s, 3H, SO₂CH₃), 1.32 (s, 9H, C(CH₃)₃) IR (r) 3352, 2962, 1513, 1336, 1123 cm⁻¹ MS m/z: 452 (MH⁺)

### EXAMPLE 20

### Preparation of N-(4-tert-butylbenzyl)-N-hydroxy-N-[3-fluoro-4-(methylsulfonylamino)benzyl]thiourea Compound (30)

The mixture of compound 19 and 3 was treated according to the same procedure described in above Example 17 to give N-(4-tert-butylbenzyl)-N-hydroxy-N-[3-fluoro-4-(methylsulfonylamino)benzyl]thiourea compound 30 (yield: 93%) (See Table 1).
melting point: 124-126° C.

¹H-NMR(CDCl₃) δ: 7.50 (t, 1H, J=8.0 Hz), 7.38 (AB q, 4H, J=8.8 Hz), 7.1-7.2 (m, 2H), 5.34 (s, 2H, CH₂NOH), 4.85 (d, 2H, J=5.6 Hz, CH₂NH), 3.00 (s, 3H, SO₂CH₃), 1.32 (s, 9H, C(CH₃)₃) IR (KBr): 3260, 2963, 1513, 1326, 1153, 1107 cm⁻¹ MS m/z: 440 (MH⁺)

### EXAMPLE 21

### Preparation of N-(4-tert-butylbenzyl)-N-hydroxy-N-[3-chloro-4-(methylsulfonylamino)benzyl]thiourea Compound (31)

The mixture of compound 20 and 3 was treated according to the same procedure described in above Example 17 to give N-(4-tert-butylbenzyl)-N-hydroxy-N-[3-chloro-4-(methylsulfonylamino)benzyl]thiourea compound 31 (yield: 91%) (See Table 1).
melting point: 119.5-122.5° C.

¹H-NMR(CDCl₃) δ: 7.62 (d, 1H, J=8.5 Hz), 7.44 (d, 1H, J=2.0 Hz), 7.36-7.42 (m, 3H), 7.26 (m, 2H), 5.36 (s, 2H, HONCH₂), 4.86 (d, 2H, J=5.8 Hz, NHCH₂), 3.01 (s, 3H, SO₂CH₃), 1.32 (s, 9H, C(CH₃)₃). IR (KBr): 3400, 2919, 1737, 1383, 1216, 1107 cm⁻¹ MS m/z 456 (MH⁺)

### EXAMPLE 22

### Preparation of N-(4-tert-butylbenzyl)-N-hydroxy-N-[4-(methylsulfonylamino)-3-nitrobenzyl]thiourea Compound (32)

The mixture of compound 21 and 3 was treated according to the same procedure described in above Example 17 to give N-(4-tert-butylbenzyl)-N-hydroxy-N-[4-(methylsulfonylamino)-3-nitrobenzyl]thiourea compound 32 (yield: 90%)(See Table 1).
melting point: 102-105° C.

¹H-NMR (CDCl₃) δ: 8.22 (d, 1H, J=2.0 Hz, ArH-2), 7.86 (d, 1H, J=8.3 Hz, ArH-5), 7.70 (dd, 1H, J=2.0, 8.3 Hz, ArH-6), 7.40 (dd, 4H, Ar), 5.36 (s, 2H, HONCH₂), 4.92 (d, 2H, J=5.6 Hz, NHCH₂), 3.14 (s, 3H, SO₂CH₃), 1.32 (s, 9H, C(CH₃)₃) IR (KBr) 3360, 2919, 1538, 1337, 1143 cm⁻¹ MS m/z: 467 (MH⁺)

### EXAMPLE 23

### Preparation of N-(4-tert-butylbenzyl)-N-hydroxy-N-[2-fluoro-4-(methylsulfonylamino)benzyl]thiourea Compound (33)

The mixture of compound 22 and 3 was treated according to the same procedure described in above Example 17 to give N-(4-tert-butylbenzyl)-N-hydroxy-N-[2-fluoro-4-(methylsulfonylamino)benzyl]thiourea compound 33 (yield: 96%) (See Table 1).
melting point: 136-137° C.

¹H-NMR(CDCl₃) δ: 7.44 (t, 1H, J=8.3 Hz), 7.38 (AB q, 4H), 7.01 (dd, 1H, J=11.2, 2.2 Hz), 6.86 (dd, 1H, J=8.3, 2.2 Hz), 6.52 (s, 1H, NHSO₂), 5.75 (s, 1H, NH), 5.32 (s, 2H, CH₂NOH), 4.87 (d, 2H, J=5.8 Hz, CH₂NH), 3.00 (s, 3H, SO₂CH₃), 1.31 (s, 9H, C(CH₃)₃). IR (KBr): 3266, 2962, 1532, 1325, 1148, 1109 cm⁻¹ MS m/z: 440 (MH⁺)

### EXAMPLE 24

### Preparation of N-(4-tert-butylbenzyl)-N-hydroxy-N-[2-chloro-4-(methylsulfonylamino)benzyl]thiourea Compound (34)

The mixture of compound 23 and 3 was treated according to the same procedure described in above Example 17 to give N-(4-tert-butylbenzyl)-N-hydroxy-N-[2-chloro-4-(methylsulfonylamino)benzyl]thiourea compound 34 (yield: 95%) See Table 1).
melting point: 150-152° C.

¹H-NMR(CDCl₃) δ: 7.50 (d, 1H, J=8.5 Hz), 7.35 (dd, 4H, J=3.4, 12.2 Hz), 7.29 (d, 1H, J=2.2 Hz), 7.04 (dd, 1H, J=8.3 and 2.2 Hz), 5.32 (s, 2H, HONCH₂), 4.92 (d, 2H, J=6.1 Hz, NHCH₂), 3.02 (s, 3H, SO₂CH3), 1.31 (s, 9H, C(CH₃)₃) IR (KBr): 3400, 2919, 1737, 1383, 1216, 1107 cm⁻¹ MS m/z: 456 (MH⁺)

TABLE 1

| | |
|---|---|
| | (VII) |
| 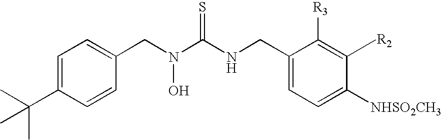 | |

| | | | | Yield | |
|---|---|---|---|---|---|
| Group | Compound | R₂ | R₃ | (%) | Spectrum data |
| III | 28 | H | H | 94 | ¹H-NMR(CDCl₃) δ: 7.38 (s, 4 H), 7.32 (d, 2 H, |
| | | | | | J = 8.3 Hz), 7.15 (d, 2 H, J = 8.3 Hz), 6.46 (s, 1 |
| | | | | | H, NHSO₂), 5.97 (bs, 1 H, NHCS), 5.34 (s, 2 H, |
| | | | | | CH₂NOH), 4.82 (d, 2 H, J = 5.6 Hz, NHCH₂), |
| | | | | | 2.97 (s, 3 H, SO₂CH₃), 1.31 (s, 9 H, C(CH₃)₃) |
| | 29 | OCH₃ | H | 92 | ¹H-NMR(CDCl₃) δ: 7.39 (m, 4 H), 6.99 (m, 1H), |
| | | | | | 6.91 (m, 1 H), 6.74 (m, 1 H), 5.52 (bs, 1 H, |
| | | | | | NH), 5.36 (s, 2 H, CH₂NHOH), 4.83 (d, 2 H, J = |
| | | | | | 5.6 Hz, CH₂NH), 3.88 (s, 3 H, OCH₃), 2.94 |
| | | | | | (s, 3 H, SO₂CH₃), 1.32 (s, 9 H, C(CH₃)₃) |
| | 30 | F | H | 93 | ¹H-NMR(CDCl₃) δ: 7.50 (t, 1 H, J = 8.0 Hz), |
| | | | | | 7.38 (AB q, 4 H, J = 8.8 Hz), 7.1-7.2 (m, 2 H), |
| | | | | | 5.34 (s, 2 H, CH₂NOH), 4.85 (d, 2 H, J = 5.6 |
| | | | | | Hz, CH₂NH), 3.00 (s, 3 H, SO₂CH₃), 1.32 (s, 9 |
| | | | | | H, C(CH₃)₃) |
| | 31 | Cl | H | 91 | ¹H-NMR(CDCl₃) δ: 7.62 (d, 1 H, J = 8.5 Hz), |
| | | | | | 7.44 (d, 1 H, J = 2.0 Hz), 7.36-7.42 (m, 3 H), |
| | | | | | 7.26 (m, 2 H), 5.36 (s, 2 H, HONCH₂), 4.86 (d, |
| | | | | | 2 H, J = 5.8 Hz, NHCH₂), 3.01 (s, 3 H, |
| | | | | | SO₂CH₃), 1.32 (s, 9 H, C(CH₃)₃) |
| | 32 | NO₂ | H | 90 | ¹H-NMR(CDCl₃) δ: 8.22 (d, 1 H, J = 2.0 Hz, |
| | | | | | ArH-2), 7.86 (d, 1 H, J = 8.3 Hz, ArH-5), 7.70 |
| | | | | | (dd, 1 H, J = 2.0, 8.3 Hz, ArH-6), 7.40 (dd, 4 H, |
| | | | | | Ar), 5.36 (s, 2 H, HONCH₂), 4.92 (d, 2 H, J = |
| | | | | | 5.6 Hz, NHCH₂), 3.14 (s, 3 H, SO₂CH₃), 1.32 |
| | | | | | (s, 9 H, C(CH₃)₃) |
| | 33 | H | F | 96 | ¹H-NMR(CDCl₃) δ: 7.44 (t, 1 H, J = 8.3 Hz), |
| | | | | | 7.38 (AB q, 4 H), 7.01 (dd, 1 H, J = 11.2, 2.2 |
| | | | | | Hz), 6.86 (dd, 1 H, J = 8.3, 2.2 Hz), 6.52 (s, 1 |
| | | | | | H, NHSO₂), 5.75 (s, 1 H, NH), 5.32 (s, 2 H, |
| | | | | | CH₂NOH), 4.87 (d, 2 H, J = 5.8 Hz, CH₂NH), |
| | | | | | 3.00 (s, 3 H, SO₂CH₃), 1.31 (s, 9 H, |
| | | | | | C(CH₃)₃) |
| | 34 | H | Cl | 95 | ¹H-NMR(CDCl₃) δ: 7.50 (d, 1 H, J = 8.5 Hz), |
| | | | | | 7.35 (dd, 4 H, J = 3.4, 12.2 Hz), 7.29 (d, 1 H, J = |
| | | | | | 2.2 Hz), 7.04 (dd, 1 H, J = 8.3 and 2.2 Hz), |
| | | | | | 5.32 (s, 2 H, HONCH₂), 4.92 (d, 2 H, J = 6.1 |
| | | | | | Hz, NHCH₂), 3.02 (s, 3 H, SO₂CH₃), 1.31 (s, 9H, |
| | | | | | C(CH₃)₃) |

### EXAMPLE 25

### Preparation of N-[2-(3,4-dimethylbenzyl)-3-(pivaloyloxy)propyl]-N-hydroxy-N-[4-(methylsulfonylamino)benzyl]thiourea Compound (35)

The mixture of compound 17 and 8 was treated according to the same procedure described in above Example 17 to give white solid of N-[2-(3,4-dimethylbenzyl)-3-(pivaloyloxy) propyl]-N-hydroxy-N-[4-(methylsulfonylamino)benzyl]thiourea compound 35 (yield: 94%) (See Table 2).
melting point: 120-123° C.

¹H-NMR(CDCl₃) δ: 7.63 (bs, 1H, NH), 7.28 (d, 2H, J=8.3 Hz), 7.15 (d, 2H, J=8.3 Hz), 6.8-7.1 (m, 4H, Ph and NH), 4.74 (d, 2H, J=5.6 Hz, NHCH₂A), 3.95-4.25 (m, 4H, CH₂OCO, CH₂NOH), 2.96 (s, 3H, SO₂CH₃), 2.5-2.75 (m, 3H, CHCH₂A), 2.24 (d, 6H, 2×CH₃), 1.20 (s, 9H, C(CH₃)₃) IR (KBr): 3266, 1698, 1539, 1337, 1154 cm⁻¹ Mass m/z: 536 (MH)

### EXAMPLE 26

### Preparation of N-[2-(3,4-dimethylbenzyl)-3-(pivaloyloxy)propyl]-N-hydroxy-N-[3-methoxy-4-(methylsulfonylamino)benzyl]thiourea Compound (36)

The mixture of compound 18 and 8 was treated according to the same procedure described in above Example 17 to give N-[2-(3,4-dimethylbenzyl)-3-(pivaloyloxy)propyl]-N-hydroxy-N-[3-methoxy-4-(methylsulfonylamino)benzyl] thiourea compound 36 (yield: 90%) (See Table 2).

¹H-NMR(CDCl₃) δ: 7.47 (d, 1H, J=8.0 Hz), 6.88-7.06 (m, 5H), 6.74 (s, 1H, NHSO₂), 4.77 (d, 2H, CH₂NOH), 4.1-4.25 (m, 3H, CH₂NH and CH₂OCO), 4.00 (AB q, 1H, J=5.4 Hz, CH₂OCO), 3.87 (s, 3H, OCH₃), 2.94 (s, 3H, SO₂CH₃), 2.5-2.7 (m, 3H, CH₂A and CH), 2.2-2.3 (m, 6H, 2×CH₃), 1.18 (s, 9H, C(CH₃)₃) IR (KBr): 3334, 2921, 1716 cm⁻¹ MS m/z: 566 (MH⁺)

### EXAMPLE 27

### Preparation of N-[2-(3,4-dimethylbenzyl)-3-(pivaloyloxy)propyl]-N-hydroxy-N-[3-fluoro-4-(methylsulfonylamino)benzyl]thiourea Compound (37)

The mixture of compound 19 and 8 was treated according to the same procedure described in above Example 17 to give N-[2-(3,4-dimethylbenzyl)-3-(pivaloyloxy)propyl]-N-hydroxy-N-[3-fluoro-4-(methylsulfonylamino)benzyl]thiourea compound 37 (yield: 93%) (See Table 2).
melting point: 52-55° C.

¹H-NMR(CDCl₃) δ: 7.74 (bs, 1H), 7.64 (bs, 1H), 7.52 (t, 1H, J=8.3 Hz), 6.9-7.25 (m, 5H), 6.45 (bs, 1H, NHSO₂), 4.81 (d, 2H, J=3.7 Hz, NHCH₂A), 4.18 (m, 3H, CH₂NOH and CH₂OCO), 4.00 (dd, 1H. CH₂OCO), 3.01 (s, 3H, SO₂CH₃), 2.5-2.8 (m, 3H, CHCH₂Ph), 2.2-2.3 (m, 6H, 2×CH₃), 1.19 (s, 9H, C(CH₃)₃) IR (KBr): 3362, 2971, 1715, 1508, 1337, 1158 cm⁻¹ MS m/z: 554 (MH⁺)

### EXAMPLE 28

### Preparation of N-[2-(3,4-dimethylbenzyl)-3-(pivaloyloxy)propyl]-N-hydroxy-N-[2-fluoro-4-(methylsulfonylamino)benzyl]thiourea Compound (38)

The mixture of compound 22 and 8 was treated according to the same procedure described in above Example 17 to give N-[2-(3,4-dimethylbenzyl)-3-(pivaloyloxy)propyl]-N-hydroxy-N-[2-fluoro-4-(methylsulfonylamino)benzyl]thiourea compound 38 (yield: 91%) (I Table 2).
melting point: 55-57° C.

¹H-NMR(CDCl₃) δ: 7.39 (t, 1H, J=8.0 Hz), 7.85-7.05 (m, 5H), 6.9-7.25 (m, 5H), 4.81 (d, 2H, J=5.6 Hz, NHCH₂A), 3.95-4.25 (m, 4H, CH₂NOH and CH₂OCO), 3.00 (s, 3H, SO₂CH₃), 2.5-2.8 (m, 3H, CHCH₂Ph), 2.2-2.3 (m, 6H, 2×CH₃), 1.19 (s, 9H, C(CH₃)₃) IR (KBr): 3254, 2971, 1701, 1626, 1530, 1331, 1149 cm⁻¹ MS m/z: 554 (MH⁺)

### EXAMPLE 29

### Preparation of N-[2-(3,4-dimethylbenzyl)-3-(pivaloyloxy)propyl]-N-hydroxy-N-[2-chloro-4-(methylsulfonylamino)benzyl]thiourea Compound (39)

The mixture of compound 23 and 8 was treated according to the same procedure described in above Example 17 to give N-[2-(3,4-dimethylbenzyl)-3-(pivaloyloxy)propyl]-N-hydroxy-N-[2-chloro-4-(methylsulfonylamino)benzyl]thiourea compound 39 (yield: 94%) (L Table 2).
melting point: 56-58° C.

¹H-NMR(CDCl₃) δ: 7.35-7.45 (m, 2H), 6.9-7.05 (m, 4H), 4.85 (d, 2H, J=6.1 Hz, NHCH₂A), 3.95-4.25 (m, 4H, CH₂NOH and CH₂OCO), 2.99 (s, 3H, SO₂CH₃), 2.5-2.8 (m, 3H, CHCH₂Ph), 2.2-2.3 (m, 6H, 2×CH₃), 1.20 (s, 9H, C(CH₃)₃) IR (KBr): 3262, 2972, 1698, 1608, 1531, 1325, 1156 cm⁻¹ MS m/z: 570 (MH⁺)

TABLE 2

| | |
|---|---|
| | (VIII) |
| 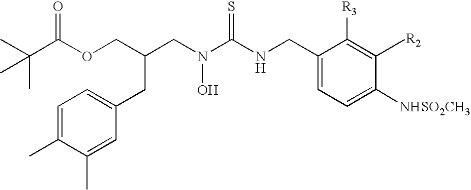 | |

| | | | | Yield | |
|---|---|---|---|---|---|
| Group | Compound | R₂ | R₃ | (%) | Spectrum data |
| III | 35 | H | H | 94 | ¹H-NMR(CDCl₃) δ: 7.63 (bs, 1 H, NH), 7.28 |
| | | | | | (d, 2 H, J = 8.3 Hz), 7.15 (d, 2 H, J = 8.3 Hz), |
| | | | | | 6.8-7.1 (m, 4 H, Ph and NH), 4.74 (d, 2 H, J = |
| | | | | | 5.6 Hz. NHCH₂Ar), 3.95-4.25 (m, 4 H, |
| | | | | | CH₂OCO, CH₂NOH), 2.96 (s, 3 H, SO₂CH₃), |
| | | | | | 2.5-2.75 (m, 3 H, CHCH₂Ar), 2.24 (d, 6 H, 2 × |
| | | | | | CH₃), 1.20 (s, 9 H, C(CH₃)₃) |
| | 36 | OCH₃ | H | 90 | ¹H-NMR(CDCl₃) δ: 7.47 (d, 1 H, J = 8.0 Hz), |
| | | | | | 6.88-7.06 (m, 5 H), 6.74 (s, 1 H, NHSO₂), |
| | | | | | 4.77 (d, 2 H, CH₂NOH), 4.1-4.25 (m, 3 H, |
| | | | | | CH₂NH and CH₂OCO), 4.00 (AB q, 1 H, J = |
| | | | | | 5.4 Hz, CH₂OCO), 3.87 (s, 3 H, OCH₃), 2.94 |
| | | | | | (s, 3 H, SO₂CH₃), 2.5-2.7 (m, 3 H, CH₂Ar and |
| | | | | | CH), 2.2-2.3 (m, 6 H, 2 × CH₃), 1.18 (s, 9 H, |
| | | | | | C(CH₃)₃) |
| | 37 | F | H | 93 | ¹H-NMR(CDCl₃) δ: 7.74 (bs, 1H), 7.64 (bs, 1H), |
| | | | | | 7.52 (t, 1 H, J = 8.3 Hz), 6.9-7.25 (m, 5H), |
| | | | | | 6.45 (bs, 1 H, NHSO₂), 4.81 (d, 2 H, J = |
| | | | | | 3.7 Hz, NHCH₂Ar), 4.18 (m, 3 H, CH₂NOH |
| | | | | | and CH₂OCO), 4.00 (dd, 1 H, CH₂OCO), 3.01 |
| | | | | | (s, 3 H, SO₂CH₃), 2.5-2.8 (m, 3 H, |
| | | | | | CHCH₂Ph), 2.2-2.3 (m, 6 H, 2 × CH₃), 1.19 |
| | | | | | (s, 9 H, C(CH₃)₃) |
| | 38 | H | F | 91 | ¹H-NMR(CDCl₃) δ: 7.39 (t, 1 H, J = 8.0 Hz), |
| | | | | | 7.85-7.05 (m, 5 H), 6.9-7.25 (m, 5 H), 4.81 (d, |
| | | | | | 2 H, J = 5.6 Hz, NHCH₂Ar), 3.95-4.25 (m, 4 |
| | | | | | H, CH₂NOH and CH₂OCO), 3.00 (s, 3 H, |
| | | | | | SO₂CH₃), 2.5-2.8 (m, 3 H, CHCH₂Ph), 2.2-2.3 |
| | | | | | (m, 6 H, 2 × CH₃), 1.19 (s, 9 H, C(CH₃)₃) |
| | 39 | H | Cl | 94 | ¹H-NMR(CDCl₃) δ: 7.35-7.45 (m, 2 H), 6.9- |
| | | | | | 7.05 (m, 4 H), 4.85 (d, 2 H, J = 6.1 Hz, |
| | | | | | NHCH₂Ar), 3.95-4.25 (m, 4 H, CH₂NOH and |
| | | | | | CH₂OCO), 2.99 (s, 3 H, SO₂CH₃), 2.5-2.8 (m, |
| | | | | | 3 H, CHCH₂Ph), 2.2-2.3 (m, 6 H, 2 × CH₃), |
| | | | | | 1.20 (s, 9 H, C(CH₃)₃) |

### EXAMPLE 30

### Preparation of N-[2-(4-tert-butylbenzyl)-3-(pivaloyloxy)propyl]-N-hydroxy-N-[4-(methylsulfonylamino)benzyl]thiourea Compound (40, SU-552)

The mixture of compound 17 and 9 was treated according to the same procedure described in above Example 17 to give white solid of N-[2-(4-tert-butylbenzyl)-3-(pivaloyloxy)propyl]-N-hydroxy-N-[4-(methylsulfonylamino)benzyl]thiourea compound 40 (yield: 97%)(gee Table 3).
melting point: 149-150° C.

¹H-NMR(CDCl₃) δ: 7.79 (bs, 1H, OH), 7.25-7.32 (m, 4H), 7.1-7.18 (m, 4H, Ar), 6.91 (bs, 1H, NHSO₂), 4.75 (d, 2H, J=5.5 Hz, NHCH₂A), 4.29 (dd of AB, 1H, J=10.3, 14.5 Hz, CH₂NOH), 4.12 (m, 2H, CH₂OCO), 3.98 (dd of AB, 1H, J=5, 14.5 Hz, CH₂NOH), 2.96 (s, 3H, SO₂CH₃), 2.69 (d, 2H, J=7 Hz, CH₂A), 2.59 (bs, 1H, CH), 1.29 (s, 9H, C(CH₃)₃), 1.16 (s, 9H, C(CH₃)₃) IR (KBr): 3295, 3186, 2964, 1706, 1529, 1321, 1184, 1147 cm⁻¹ MS m/z: 564 (MH⁺)

### EXAMPLE 31

### Preparation of N-[2-(4-tert-butylbenzyl)-3-(pivaloyloxy)propyl]-N-hydroxy-N-[3-fluoro-4-(methylsulfonylamino)benzyl]thiourea Compound (41)

The mixture of compound 19 and 9 was treated according to the same procedure described in above Example 17 to give white solid of N-[2-(4-tert-butylbenzyl)-3-(pivaloyloxy)propyl]-N-hydroxy-N-[3-fluoro-4-(methylsulfonylamino)benzyl]thiourea compound 41 (yield: 95%)(See Table 3).
melting point: 128-129° C.

¹H-NMR(CDCl₃) δ: 7.83 (bs, 1H), 7.49 (t, 1H, J=8.0 Hz), 7.31 (d, 2H, J=8.3 Hz), 7.05-7.2 (m, 3H), 6.60 (bs, 1H, NHSO₂), 4.79 (m, 2H, NHCH₂A), 4.29 (dd, 1H, CH₂OCO), 4.05-4.20 (m, 2H, CH₂NOH), 3.97 (dd, 1H, CH₂OCO), 3.00 (s, 3H, SO₂CH₃), 2.69 (d, 2H, J=7.1 Hz, CH₂A), 2.58 (bs, 1H, CH), 1.29 (s, 9H, C(CH₃)₃), 1.16 (s, 9H, C(CH₃)₃) IR (KBr): 3244, 2964, 1716, 1509, 1331, 1158 cm⁻¹ MS m/z: 582 (MH⁺)

TABLE 3

| | |
|---|---|
| | (IX) |
| 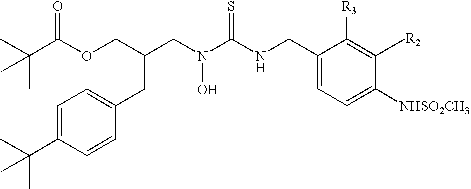 | |

| Group | Compound | R₂ | R₃ | Yield (%) | Spectrum data |
|---|---|---|---|---|---|
| IV | 40 | H | H | 97 | ¹H-NMR(CDCl₃) δ: 7.79 (bs, 1 H, OH), |
| | | | | | 7.25-7.32 (m, 4 H), 7.1-7.18 (m, 4 H, Ar), |
| | | | | | 6.91 (bs, 1 H, NHSO₂), 4.75 (d, 2 H, J = 5.5 |
| | | | | | Hz, NHCH₂Ar), 4.29 (dd of AB, 1 H, J = |
| | | | | | 10.3, 14.5 Hz, CH₂NOH), 4.12 (m, 2 H, |
| | | | | | CCH₂OCO), 3.98 (dd of AB, 1 H, J = 5, 14.5 |
| | | | | | Hz, CH₂NOH), 2.96 (s, 3 H, SO₂CH₃), 2.69 |
| | | | | | (d, 2 H, J = 7 Hz, CH₂Ar), 2.59 (bs, 1 H, |
| | | | | | CH), 1.29 (s, 9 H, 0(CH₃)₃), 1.16 (s, 9 H, |
| | | | | | C(CH₃)₃) |
| | 41 | F | H | 95 | ¹H-NMR(CDCl₃) δ: 7.83 (bs, 1 H), 7.49 (t, 1 |
| | | | | | H, J = 8.0 Hz), 7.31 (d, 2 H, J = 8.3 Hz), |
| | | | | | 7.05-7.2 (m, 3 H), 6.60 (bs, 1 H, NHSO₂), |
| | | | | | 4.79 (m, 2 H, NHCH₂Ar), 4.29 (dd, 1 H, |
| | | | | | CH₂OCO), 4.05-4.20 (m, 2 H, CH₂NOH), |
| | | | | | 3.97 (dd, 1 H, CH₂OCO), 3.00 (s, 3 H, |
| | | | | | SO₂CH₃), 2.69 (d, 2 H, J = 7.1 Hz, CH₂Ar), |
| | | | | | 2.58 (bs, 1 H, CH), 1.29 (s, 9 H, C(CH₃)₃), |
| | | | | | 1.16 (s, 9 H, C(CH₃)₃) |

### EXAMPLE 32

### Preparation of 4-(methylsulfonylamino)phenyl acetic acid Compound (43)

A solution of 4-aminophenylacetic acid (1 g, 6.66 mmol) in THF (10 ml) was adjusted to pH 9 with 1 N sodium hydroxide. The mixture was reacted by the dropwise addition of methansulfonyl chloride (0.77 ml, 9.99 mmol) in THF (10 ml), adjusted to pH 3 with 1 N hydrochloric acid, diluted with distilled water and extracted with ethyl acetate several times.

The combined organic layers were washed with water, dried over MgSO₄ and concentrated in vacuo. The residue was purified by flash column chromatography on Silica gel with EtOAc/hexanes (2:3) solvent mixture as an eluant to give 0.855 g of yellow solid of 4-(methylsulfonylamino)phenylacetic acid compound 43 (yield: 56%).

¹H-NMR(DMSO-d₆) δ: 9.67 (s, 1H, COOH), 7.20 (d, 2H, J=8.5 Hz, Ar), 7.13 (d, 2H, J=8.5 Hz, Ar), 3.50 (s, 2H, CH₂), 3.95 (s, 3H, SO₂CH₃)

### EXAMPLE 33

### Preparation of pentafluorophenyl 2-[4-(methylsulfonylamino)phenyl] acetate Compound (44)

A cooled solution of 0.6707 g of pentafluoro phenol (3.3 mmol) and 0.036 g of dimethylaminopyridine (0.3 mmol) in dichloromethane (15 ml) was reacted by the dropwise addition of 4.5 mg of 1.0M dicyclohexyl carboimide and stirred for 16 hours at room temperature. The reaction mixture was concentrated in vacuo, diluted with ether, filtered and the filtrate was concentrated again in vacuo. The residue was purified by column chromatography with EtOAc/hexanes (1:10) solvent mixture as an eluant to give 0.592 g of white solid of pentafluorophenyl 2-[4-(methylsulfonylamino)phenyl] acetate compound 44 (yield: 50%).

¹H-NMR(CDCl₃) δ: 7.36 (d, 2H, J=8.5 Hz, Ar), 7.24 (d, 2H, J=8.5 Hz, Ar), 3.96 (s, 2H, CH₂), 3.03 (s, 3H, SO₂CH₃).

### EXAMPLE 34

### Preparation of N-(4-tert-butylbenzyl)-N-hydroxy-[4-(methylsulfonylamino)phenyl] acetamide Compound (45)

The mixture of compound 44 and 3 was condensed according to the same procedure described in above Example 33 to give white solid of N-(4-tert-butylbenzyl)-N-hydroxy-[4-(methylsulfonylamino)phenyl]acetamide compound 45 (yield: 47%) See Table 4).
melting point: 161-163° C.

¹H-NMR(acetone-d₆) δ: 9.02 (bs, 1H, OH), 8.48 (bs, 1H, NHSO₂), 7.2-7.4 (m, 8H, Ar), 4.75 (s, 2H, CH₂NOH), 3.82 (s, 2H, CH₂CO), 2.95 (s, 3H, SO₂CH₃), 1.29 (s, 9 H, C(CH₃)₃) IR (KBr): 3350, 1650, 1515, 1338, 1154 cm⁻¹ MS m/z: 391 (MH⁺)

TABLE 4

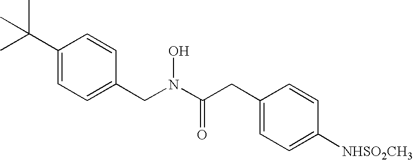

| Group | Compound | Yield (%) | Spectrum data |
|---|---|---|---|
| IV | 45 | 47 | ¹H-NMR(acetone-d₆) δ: 9.02 (bs, 1 H, OH), 8.48 (bs, 1 H, |
| | | | NHSO₂), 7.2-7.4 (m, 8 H, Ar), 4.75 (s, 2 H, CH₂NOH), |
| | | | 3.82 (s, 2 H, CH₂CO), 2.95 (s, 3 H, SO₂CH₃), 1.29 (s, 9 H, |
| | | | C(CH₃)₃) |

### EXAMPLE 35

### Preparation of tert-butyl N-[(tert-butoxycarbonyl)oxy]-N-(4-nitrobenzyl)carbamate Compound (47)

4-nitrobenzyl bromide as a starting material was reacted with tert-butyl-N-(tert-butoxycarbonyloxy)carbamate under basic condition to give colorless oil of tert-butyl N-[(tert-butoxycarbonyl)oxy]-N-(4-nitrobenzyl)carbamate compound 47 (yield: 81%).

¹H-NMR(CDCl₃) δ: 8.14 (dt, 2H, J=2.2, 8.6 Hz, Ar), 7.48 (d, 2H, J=8.6 Hz, Ar), 4.81 (s, 2H, CH₂), 1.44 (bs, 18H, 2×C(CH₃)₃)

### EXAMPLE 36

### Preparation of tert-butyl N-[(tert-butoxycarbonyl)oxy]-N-(4-nitrobenzyl)carbamate Compound (48)

A suspension of compound 47 (6.40 g, 17.3 mmol) and Pd—C (650 mg) in MeOH (100 ml) was hydrogenated under a hydrogen balloon for 2 hrs. The reaction mixture was filtered and the filtrate was concentrated in vacuo. The residue was dissolved in 60 ml pyridine. The mixture was treated with methansulfonylchloride (20.1 ml, 26.0 mmol) and stirred for 16 hours at room temperature. The reaction mixture was concentrated in vacuo, diluted with distilled water, extracted with ethyl acetate several times.

The combined organic layers were washed with water and brine, dried over MgSO₄ and concentrated in vacuo. The residue was purified by column chromatography on Silica gel with EtOAc/hexanes (2:3) solvent mixture as an eluant to give 6.56 g of viscous syrup of tert-butyl N-[(tert-butoxycarbonyl)oxy]-N-(4-nitrobenzyl)carbamate compound 48 (yield: 91%).

¹H-NMR(CDCl₃) δ: 7.32 (d, 2H, J=8.6 Hz, Ar), 7.20 (dd, 2H, J=1.7, 8.6 Hz, Ar), 4.72 (s, 2H, CH₂), 2.99 (s, 3H, SO₂CH₃), 1.48 (bs, 18H, 2×C(CH₃)₃).

### EXAMPLE 37

### Preparation of N-[4-(methylsulfonylamino)benzyl]hydroxylamine Compound (49)

A cooled solution of the compound 48 (6.56 g, 15.7 mmol) was treated with trifluoroacetic acid (30 ml) at 0° C. and stirred for 20 mins at room temperature. The mixture was concentrated in vacuo to obtain 5.19 g of yellow solid of N-[4 (methylsulfonylamino)benzyl]hydroxylamine 49 (yield: 100%).

¹H-NMR(DMSO-d₆) δ: 11.26 (bs, 1H), 10.8 (bs, 1H), 9.87 (s, 1H), 7.34 (d, 2H, J=8.5 Hz, Ar), 7.15 (dd, 2H, J=8.5 Hz, Ar), 4.19 (s, 2H, CH₂), 2.94 (s, 3H, SO₂CH3).

### EXAMPLE 38

### Preparation of benzyl N-(2-fluoro-4-methylphenyl)carbamate Compound (51)

A solution of 2-fluoro-4-methylaniline compound 50 (400 mg, 3.2 mmol) in pyridine (4 ml) was reacted by dropwise addition of benzylchloroformate (0.68 ml, 4.8 mmol) at 0° C. After being stirred for 20 min at 0° C., the reaction was stopped by addtion of 0.2 ml ethanol. The reaction mixture was diluted with distilled water, filtered. The residue was purified by column chromatography on Silica gel with EtOAc/hexanes (1:10) solvent mixture as an eluant to give 730 mg of pale pink solid of benzyl N-(2-fluoro-4-methylphenyl)carbamate compound 51 (yield: 88%).
melting point: 66° C.

¹H-NMR(CDCl₃) δ: 7.93 (bt, 1H), 7.3-7.45 (m, 5H, Ph), 6.86-6.93 (m, 2H), 6.80 (bs, 1H, NH), 5.21 (s, 2H, OCH₂Ph), 2.30 (s, 3H, CH₃)

### EXAMPLE 39

### Preparation of benzyl N-(4-(bromomethyl)-2-fluorophenyl)carbamate Compound (52)

A solution of 500 mg of benzyl N-(2-fluoro-4-methylphenyl)carbamate compound 51 in dichloromethane (8 ml) was treated with NBS (360 mg, 2.02 mmol) and AIBN as a catalyst. The reaction mixture was refluxed under 300-watt halogen lamp for 150 mins, cooled down at room temperature and dehydrated. The residue was purified by column chromatography on Silica gel with EtOAc/hexanes (1:10) solvent mixture as an eluant to give 268 mg of dark gray solid of N-(4-(bromomethyl)-2-fluorophenyl)carbamate compound 52 (yield: 41%).
melting point: 95-96° C.

¹H-NMR (CDCl₃) δ: 8.10 (bt, 1H, J=8.4 Hz), 7.35-7.45 (m, 5H, Ph), 7.10-7.16 (m, 2H), 6.94 (bs, 1H, NH), 5.22 (s, 2H, OCH₂Ph), 4.43 (s, 2H, CH₂Br)

### EXAMPLE 40

### Preparation of tert-butyl N-[(tert-butoxycarbonyl)oxy]-N-{4-[(benzyloxy)carbonylamino]-3-fluorobenzyl}carbamate Compound (53)

A solution of tert-butyl-N-(tert-butoxycarbonyloxy)carbamate (224 mg, 0.96 mmol) in DMF (2 ml) was reacted with sodium hydride (38 mg, 0.96 mmol) at 0° C. and stirred for 20 mins at room temperature. The reaction mixture was treated by dropwise addition of benzyl N-[4-(bromomethyl)-2-fluorophenyl]carbamate compound 52 (250 mg, 0.74 mmol) and stirred for 1 hour. After concentrating, residual mixture was purified by column chromatography on Silica gel with EtOAc/hexanes (1:5) solvent mixture as an eluant to give 355 mg of yellow oil of tert-butyl N-[(tert-butoxycarbonyl)oxy]-N-{4-[(benzyloxy)carbonylamino]-3-fluorobenzyl}carbamate compound 53 (yield: 98%).

¹H-NMR(CDCl₃) δ: 8.06 (bt, 1H), 7.35-7.45 (m, 5H, Ph), 7.05-7.12 (m, 2H), 6.89 (bs, 1H, NH), 5.22 (s, 2H, OCH₂Ph), 4.68 (s, 2H, CH₂NO), 1.48 (s, 9H, C(CH₃)₃), 1.47 (s, 9H. C(CH₃)₃)

### EXAMPLE 41

### Preparation of tert-butyl N-[(4-amino-3-fluorobenzyl)-N-[(tert-butoxycarbonyl)oxy]carbamate Compound (54)

A suspension of compound 53 (350 mg, 0.714 mmol) and 10% Pd—C (35 mg) in MeOH (8 ml) was hydrogenated under a hydrogen balloon for 2 hrs at room temperature. The reaction mixture was filtered and the filtrate was concentrated in vacuo. The residue was crystallized by hexane to give 232 mg of ivory solid of tert-butyl N-[(4-amino-3-fluorobenzyl)-N-[(tert-butoxycarbonyl)oxy]carbamate compound 54 (yield: 91%).
melting point: 105-106° C.

¹H-NMR(CDCl₃) δ: 6.99 (dd, 1H, J=1.6, 12 Hz), 6.90 (dd, 1H, J=1.6, 8.1 Hz), 6.71 (t, 1H, J=8.8 Hz), 4.61 (s, 2H, CH₂NO), 3.70 (bs, 2H, NH₂), 1.48 (s, 9H, C(CH₃)₃), 1.47 (s, 9H, C(CH₃)₃)

### EXAMPLE 42

### Preparation of tert-butyl N-[(tert-butoxycarbonyl)oxy]-N-[3-fluoro-4-(methylsulfonylamino)benzyl]carbamate Compound (55, SU-576)

A cooled solution of compound 54 (210 mg, 0.59 mmol) in pyridine (2 mg) was reacted by dropwise addition of methansulfonyl chloride (0.09 ml, 1.178 mmol) at 0° C. and stirred for 30 mins at 0° C. The reaction mixture was purified by column chromatography on Silica gel with EtOAc/hexanes (1:2) solvent mixture as an eluant and crystailized by hexane and diethylester to give 238 mg of tert-butyl N-[(tert-butoxycarbonyl)oxy]-N-[3-fluoro-4-(methylsulfonylamino)benzyl]carbamate compound 55 (SU-576) (yield: 93%).
melting point: 112-113° C.

¹H-NMR (CDCl₃) δ: 7.53 (t, 1H, J=8.25 Hz), 7.12-7.2 (m, 2H), 6.90 (bs, 1H, NH), 4.73 (s, 2H, CH₂NO), 3.02 (s, 3H, SO₂CH₃), 1.49 s, 18H)

### EXAMPLE 43

### Preparation of N-[3-fluoro-4-(methylsulfonylamino)benzyl] hydroxylamine Compound (56)

A cooled solution of compound 55 (225 mg, 0.518 mmol) in dichloromethane (10 mg) was reacted with trifluoroacetic acid (2 mg) at 0° C. and stirred for 50 mins at room temperature. The reaction mixture was dehydrated below the room temperature, concentrated in vacuo. The residue was dissolved in ethyl acetate and washed with saturated sodium bicarbonate several times.

The combined organic layers were dried over MgSO₄ and concentrated in vacuo to give N-[3-fluoro-4-(methylsulfonylamino)benzyl] hydroxylamine compound 56.

¹H-NMR(CDCl₃) δ: 7.56 (m, 1H), 7.1-7.3 (m, 2H), 7.02 (bs, 1H, NHSO₂), 4.85 (s, 2 H, CH₂NOH), 2.94 (s, 3H, SO₂CH₃)

### EXAMPLE 44

### Preparation of 4-(tert-butylbenzyl)isothiocyanate Compound (57)

A cooled solution of 4-tert-butylbenzylamine (1 g, 6.13 mmol) and triethylamine (1.29 ml, 9.20 mmol) in dichloromethane (20 ml) was reacted with 1,1-thio-di-2-pyridone-1.42 g, 6.13 mmol) at 0° C., stirred for 20 mins at room temperature and concentrated in vacuo. The residue was purified by column chromatography on Silica gel with EtOAc/hexanes (1:10) solvent mixture as an eluant to give 0.755 g of white solid of 4-(tert-butylbenzyl)isothiocyanate compound 57 (yield: 60%).
melting point: 47.3° C.

¹H-NMR(CDCl₃) δ: 7.40 (dt, 2H, J=2.2, 8.6 Hz, Ar), 7.24 (d, 2H, J=8.6 Hz, Ar), 4.67 (s, 2H, CH₂), 1.32 (s, 9H, C(CH₃)₃)

### EXAMPLE 45

### Preparation of 4-(tert-butylbenzyl)isothiocyanate Compound (58)

A solution of 4-tert-butylbenzylamine (1 g, 6.13 mmol) in toluene (10 ml) was reacted with triphosgen (2.48 g, 9.20 mmol). The reaction mixture was refluxed at 100° C. for 20 mins and concentrated in vacuo. The residue was purified by column chromatography on Silica gel with EtOAc/hexanes (1:10) solvent mixture as an eluant to give 0.859 g of colorless oil of 4-(tert-butylbenzyl)isothiocyanate compound 58 (yield: 74%).

¹H-NMR(CDCl₃) δ: 7.39 (dt, 2H, J=2.2, 8.6 Hz, Ar), 7.23 (d, 2H, J=8.6 Hz, Ar), 4.43 (s, 2H, CH₂), 1.31 (s, 9H, C(CH₃)₃)

### EXAMPLE 46

### Preparation of pentafluorophenyl 2-(4-tert-butylphenyl)acetate Compound (59)

A cooled solution of 4-tert-butylphenyl acetic acid (1 g, 5.20 mmol), pentafluorophenol (1.15 g, 6.24 mmol) and dimethylaminopyridine in dichloromethane (30 ml) was reacted with 1.0 M dicyclohexylcarbodiimide (6.24 ml, 6.24 mmol) at 0° C. And the reaction mixture was stirred for 16 hours at room temperature, concentrated in vacuo, diluted with ether and filtered. The filtrate was concentrated again in vacuo and purified by column chromatography on Silica gel with EtOAc/hexanes (1:10) solvent mixture as an eluant to give 1.86 g of colorless oil of pentafluorophenyl 2-(4-tert-butylphenyl)acetate compound 59 (yield: 100%).

¹H-NMR(CDCl₃) δ: 7.40 (dt, 2H, J=2.2, 8.3 Hz, Ar), 7.28 (d, 2H, J=8.3 Hz, Ar), 3.94 (s, 2H, CH₂), 1.32 (s, 9H, C(CH₃)₃)

### EXAMPLE 47

### Preparation of N-(4-tert-butylbenzyl)-N-hydroxy-N-[4-(methylsulfonylamino)benzyl]thiourea Compound (60)

N-[4-(methylsulfonylamino)benzyl]hydroxylamine compound 49 (165 mg, 0.5 mmol) and isopropylethylamine (0.13 mg, 0.75 mmol) in DMF 3 ml was stirred for 1 hour at room temperature. The mixture was further added with above compound 57 (0.5 mmol), stirred for 20 hour at room temperature, diluted with H₂O and extracted with ethylacetate several times. The combined organic layers were washed with H₂O, dried over MgSO₄, and concentrated in vacuo. The residue was purified by column chromatography with EtOAc/hexanes (2:1) solvent mixture as an eluant to give white solid of N-(4-tert-butylbenzyl)-N-hydroxy-N-[4-(methylsulfonylamino)benzyl]thiourea compound 60 (yield: 90%) (Lee Table 5).
melting point: 124° C.

¹H-NMR(acetone-d₆) δ: 8.77 (bs, 1H, N—OH), 8.22 (t, 1H, J=6.0 Hz, NHCS), 7.25-7.45 (m, 8H), 5.34 (s, 2H, HONCH₂A), 4.84 (d, 2H, J=6.0 Hz, ArCH₂NH), 2.97 (s, 3H, SO₂CH₃), 1.29 (s, 9H, C(CH₃)₃) MS m/z: 422 (MH⁺)

### EXAMPLE 48

### Preparation of N-(4-tert-butylbenzyl)-N-hydroxy-N-[4-(methylsulfonylamino)benzyl]urea Compound (62)

N-[4-(methylsulfonylamino)benzyl]hydroxylamine compound 49 (165 mg, 0.5 mmol) and isopropylethylamine (0.13 ml, 0.75 mmol) in DMF 3 ml was stirred for 1 hour at room temperature. The mixture was further added with above compound 58 (0.5 mmol), stirred for 20 hour at room temperature, diluted with H₂O and extracted with ethylacetate several times. The combined organic layers were washed with H₂O, dried over MgSO₄, and concentrated in vacuo. The residue was purified by column chromatography with EtOAc/hexanes (2:1) solvent mixture as an eluant to give white solid of N-(4-tert-butylbenzyl)-N-hydroxy-N-[4-(methylsulfonylamino)benzyl]urea compound 62 (yield: 74%) (Lee Table 5).
melting point: 125° C.

¹H-NMR (CDCl₃) δ 7.32 (d, 2H, J=8.3 Hz), 7.27 (d, 2H, J=8.3 Hz), 7.18 (d, 2H, J=8.3 Hz), 7.10 (d, 2H, J=8.3 Hz), 6.76 (bs, 1H, NH), 6.69 (bs, 1H, OH), 6.29 (t, 1H, J=5.8 Hz, NH), 4.59 (s, 2H, HONCH₂A), 4.36 (d, 2H, J=5.8 Hz, ArCH₂NH), 2.96 (s, 3H, SO₂CH₃), 1.29 (s, 9H, C(CH₃)₃) MS m/z: 406 (MH⁺)

TABLE 5

| | |
|---|---|
| | (X) |
| 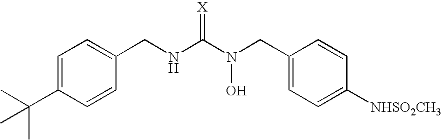 | |

| Group | Compound | X | Yield (%) | Spectrum data |
|---|---|---|---|---|
| V | 60 | S | 80 | ¹H-NMR(acetone-d₆) δ: 8.77 (bs, 1 H, N—OH), 8.22 |
| | | | | (t, 1 H, J = 6.0 Hz, NHCS), 7.25-7.45 (m, 8 H), 5.34 |
| | | | | (s, 2 H, HONCH₂Ar), 4.84 (d, 2 H, J = 6.0 Hz, |
| | | | | ArCH₂NH), 2.97 (s, 3 H, SO₂CH₃), 1.29 (s, 9 H, |
| | | | | C(CH₃)₃) |
| | 62 | O | 74 | ¹H-NMR (CDCl₃) δ 7.32 (d, 2 H, J = 8.3 Hz), 7.27 |
| | | | | (d, 2 H, J = 8.3 Hz), 7.18 (d, 2 H, J = 8.3 Hz), 7.10 |
| | | | | (d, 2 H, J = 8.3 Hz), 6.76 (bs, 1 H, NH), 6.69 (bs, 1 |
| | | | | H, OH), 6.29 (t, 1 H, J = 5.8 Hz, NH), 4.59 (s, 2 H, |
| | | | | HONCH₂Ar), 4.36 (d, 2 H, J = 5.8 Hz, ArCH₂NH), |
| | | | | 2.96 (s, 3 H, SO₂CH₃), 1.29 (s, 9 H, C(CH₃)₃) |

### EXAMPLE 49

### Preparation of N-[2-(3,4-dimethylbenzyl)-3-(pivaloyloxy)propyl]-N-hydroxy-N-[4-(methylsulfonylamino)benzyl]thiourea Compound (61)

N-[4-(methylsulfonylamino)benzyl]hydroxylamine compound 49 (165 mg, 0.5 mmol) and isopropylethylamine (0.13 nm, 0.75 mmol) in DMF 3 ml was stirred for 1 hour at room temperature. The mixture was further added with above compound 26 (0.5 mmol), stirred for 20 hour at room temperature, diluted with H₂O and extracted with ethylacetate several times. The combined organic layers were washed with H₂O, dried over MgSO₄, and concentrated in vacuo. The residue was purified by column chromatography with EtOAc/hexanes (2:1) solvent mixture as an eluant to give white solid of N-[2-(3,4-dimethylbenzyl)-3-(pivaloyloxy)-N-hydroxy-N-[4-(methylsulfonylamino)benzyl] thiourea compound 61 (yield: 35%) (See Table 6).
melting point: 49° C.

¹H-NMR(CDCl₃) δ: 7.37 (d, 2H, J=7.6 Hz), 7.14 (d, 2H, J=7.6 Hz), 6.88-7.1 (m, 3H, Ph and NH), 6.6-6.7 (bs, 2H, NH), 5.24 (m, 2H, HONHCH₂A), 4.12 (m, 1H, CH₂OCO), 3.86 (m, 1H, CH₂OCO), 3.73 (m, 1H, CH₂NH), 3.50 (m, 1H, CH₂NH), 2.97 (s, 3H, SO₂CH₃), 2.6-2.75 (m, 2H, CHCH₂A), 2.38 (m, 1H, CHCH₂A), 2.21-2.23 (d, 6H, 2×CH₃), 1.23 (s, 9H, C(CH₃)₃) IR (KBr): 3244, 1715, 1514, 1457, 1398, 1329, 1286, 1154 cm⁻¹ Mass m/z: 536 (MH⁺)

### EXAMPLE 50

### Preparation of N-[2-(3,4-dimethylbenzyl)-3-(pivaloyloxy)propyl]-N-hydroxy-N-[3-fluoro-4-(methylsulfonylamino)benzyl]thiourea Compound (64)

N-[3-fluoro-4-(methylsulfonylamino)benzyl]hydroxylamine compound 56 (165 mg, 0.5 mmol) and isopropylethylamine (0.13 ml, 0.75 mmol) in DMF 3 ml was stirred for 1 hour at room temperature. The mixture was further added with above compound 26 (0.5 mmol), stirred for 20 hour at room temperature, diluted with H₂O and extracted with ethylacetate several times. The combined organic layers were washed with H₂O, dried over MgSO₄, and concentrated in vacuo. The residue was purified by column chromatography with EtOAc/hexanes (2:1) solvent mixture as an eluant to give colorless oil of N-[2-(3,4-dimethylbenzyl)-3-(pivaloyloxy)propyl]-N-hydroxy-N-[3-fluoro-4-(methylsulfonylamino)benzyl]thiourea compound 64 (yield: 41%)(e Table 6).

¹H-NMR(CDCl₃) δ: 7.45 (t, 1H, J=8.25 Hz), 7.31 (m, 1H), 7.12-7.25 (m, 2H), 6.9-7.05 (m, 2H), 6.70 (bs, 1H, NH), 5.20 (m, 2H, CH₂NOH), 4.12 (m, 1H, CH₂OCO), 3.86 (m, 1H, CH₂OCO), 3.75 (m, 1H, CH₂NH), 3.48 (m, 1H, CH₂NH), 3.00 (s, 3H, SO₂CH3), 2.6-2.8 (m, 2H, CH₂A), 2.36 (m, 1H, CH), 2.2-2.3 (m, 6H, 2×CH₃), 1.23 (s, 9H, C(CH₃)₃), 1.22 (s, 9H, C(CH₃)₃) MS m/z: 554 (MH⁺)

TABLE 6

| | |
|---|---|
| | (XI) |
| 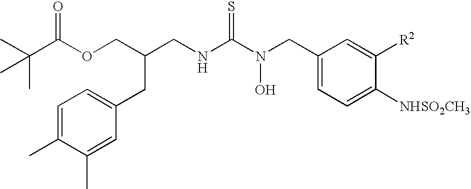 | |

| Group | Compound | R₂ | Yield (%) | Spectrum data |
|---|---|---|---|---|
| V | 61 | H | 74 | ¹H-NMR(CDCl₃) δ: 7.37 (d, 2 H, J = 7.6 Hz), 7.14 |
| | | | | (d, 2 H, J = 7.6 Hz), 6.88-7.1 (m, 3H, Ph and NH), |
| | | | | 6.6-6.7 (bs, 2 H, NH), 5.24 (m, 2 H, HONHCH₂Ar), |
| | | | | 4.12 (m, 1 H, CH₂OCO), 3.86 (m, 1 H, CH₂OCO), |
| | | | | 3.73 (m, 1 H, CH₂NH), 3.50 (m, 1 H, CH₂NH), 2.97 |
| | | | | (s, 3 H, SO₂CH₃), 2.6-2.75 (m, 2 H, CHCH₂Ar), 2.38 |
| | | | | (m, 1 H CHCH₂Ar), 2.21-2.23 (d, 6 H, 2 × Ch₃), |
| | | | | 1.23 (s, 9 H, C(CH₃)₃) |
| | 64 | F | 41 | ¹H-NMR(CDCl₃) δ: 7.45 (t, 1 H, J = 8.25 Hz), |
| | | | | 7.31 (m, 1 H), 7.12-7.25 (m, 2 H), 6.9-7.05 (m, |
| | | | | 2 H), 6.70 (bs, 1 H, NH), 5.20 (m, 2 H, |
| | | | | CH₂NOH), 4.12 (m, 1 H, CH₂OCO), 3.86 (m, 1 |
| | | | | H, CH₂OCO), 3.75 (m, 1 H, CH₂NH), 3.48 (m, |
| | | | | 1 H, CH₂NH), 3.00 (s, 3 H, SO₂CH₃), 2.6-2.8 |
| | | | | (m, 2 H, CH₂Ar), 2.36 (m, 1 H, CH), 2.2-2.3 (m, |
| | | | | 6 H, 2 × CH₃), 1.23 (s, 9 H, C(CH₃)₃), 1.22 (s, 9 |
| | | | | H, C(CH₃)₃) |

### EXAMPLE 51

### Preparation of N-hydroxy-N-[4-(methylsulfonylamino)benzyl]-2-(4-tert-butylphenyl)acetamide Compound (63)

N-[4-(methylsulfonylamino)benzyl]hydroxylamine compound 49 (165 mg, 0.5 mmol) and isopropylethylamine (0.13 ml, 0.75 mmol) in DMF 3 ml was stirred for 1 hour at room temperature. The mixture was further added with above compound 59 (0.5 mmol), stirred for 20 hours at room temperature, diluted with H₂O and extracted with ethylacetate several times. The combined organic layers were washed with H₂O, dried over MgSO₄, and concentrated in vacuo. The residue was purified by column chromatography with EtOAc/hexanes (2:1) solvent mixture as an eluant to give white solid of N-hydroxy-N-[4-(methylsulfonylamino)benzyl]-2-(4-tert-butylphenyl)acetamide compound 63 (yield: 38%) (See Table 7).

¹H-NMR(acetone-d₆) δ 7.32 (d, 2H, J=8.3 Hz), 7.25 (s, 4H), 7.21 (d, 2H, J=8.3 Hz), 4.76 (s, 2H, HONCH₂A), 3.80 (s, 2H, ArCH₂CO), 2.96 (s, 3H, SO₂CH₃), 1.28 (s, 9H. C(CH₃)₃) MS m/z: 391 (MH⁺)

TABLE 7

| | |
|---|---|
| | (XI) |
| 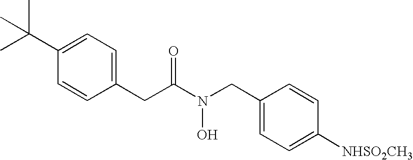 | |

| Group | Compound | Yield (%) | Spectrum data |
|---|---|---|---|
| VI | 63 | 38 | ¹H-NMR(acetone-d₆) δ 7.32 (d, 2 H, J = 8.3 Hz), 7.25 |
| | | | (s, 4 H), 7.21 (d, 2 H, J = 8.3 Hz), 4.76 (s, 2 H, |
| | | | HONCH₂Ar), 3.80 (s, 2 H, ArCH₂CO), 2.96 (s, 3 H, |
| | | | SO₂CH₃), 1.28 (s, 9 H, C(CH₃)₃) |

### REFERENCE EXAMPLE 1

### Vanilloid Receptor Binding Affinity Assay

The binding affinity activity of the target compounds for vanilloid receptor-1 was measured by an in vitro receptor binding affinity assay. In the receptor binding assay, the compounds were evaluated for their ability to displace bound [³H]RTX from the receptor. The results are expressed in terms of Ki values (mean±SEM, 3 experiments) which represent the concentration of the non-radioactive ligand that displaces half of the bound labeled RTX.

Cell Culture Preparation

The VR receptor binding affinity activity of the inventive compounds was measured by using Chinese Hamster Ovary (CHO, ATCC, No. CCL-61) cell whose cDNA of VR1 (pUHG102 VR1 plasmid) was transfected, which can control the expression of VR1 according to the presence of tetracycline and Tetracycline on/off system (pTet off regulatory plasmid, Clontech. Inc., USA) that the expression of VR1 is induced by removing tetracycline from the medium. CHO cells were cultured in the medium containing 1 μg/ml of tetracycline (T-7660, Sigma-Aldrich. Co., USA) and 10 ng/mg of puromycin for stabilizing the cell line. The cells were cultured after removing tetracycline prior to 48 hours. The tetracycline free culture medium was seeded at the bottom of T75 flask, incubated to the extent that its density reaches at 90%, and washed once with PBS buffer solution. The cells were collected by using saline solution containing 5 mM EDTA and subjected to centrifugation slightly to obtain precipitates, further, which had been kept at the temperature of −20° C. before use.

Resiniferatoxin (RTX) Competition Binding Assay.

[3H] RTX binding assay of present invention was performed with the procedure described in the literature (Szallasi et al.; Pharmacol. Exp. Ther, 262, pp 883-888, 1992).

Experiments were designed to assess inhibition of specific [³H]RTX binding to membranes by non-radioactive compounds. The binding assay mixture containing [³H]RTX (80 μM), various concentrations of competitive binding substances, 0.25 mg/ml of BSA (Cohn fraction V), 5×10⁴ ˜5×10⁵ numbers of VR1 and the expression cell, was admixed with saline solution containing 450 μl of Ca²⁺ and Mg²⁺ and 0.25 mg/W of BSA. Non-specific binding assay was measured after mixing 100 nM of non-radioactive RTX thereto. The reaction mixture was treated for 60 min at 37° C. and the reaction was quenched by cooling over ice. RTX bound to the membrane of VR1 was subjected to centrifugation with maximum velocity for 15 minutes to precipitate its membrane residue, which results in separating from non-binding RTX. The tips of tube containing above precipitate was cut off and the amount of bound radioisotope was determined by scintillation counter (LS6500, Beckman-Coulter, USA). The measurement of binding was determined in triplicate in each experiment, and each experiment was repeated at least two times. Binding data were analyzed by fitting to the Hill equation and the Ki (equilibrium binding parameter) index, the Bmax (maximum binding parameter) index, and the cooperativity index etc., were determined by using origin 6.0 program (Origin, MicroCal Co., USA).

The Preparation of Sample

An initial compound was dissolved in DMSO (dimethyl sulfoxide) and diluted with saline solution containing Ca²⁺ and Me⁺, and 0.25 mg/μl of BSA.

### EXPERIMENTAL EXAMPLE 1

### ⁴⁵Ca Influx Test

The ⁴⁵Ca Influx test by using CHO cells expressing VR1 was performed by the procedure described in the literature (Lee, J. W., Bioorganic & Medicinal Chemistry, pp 1713-1720, 2001).

The ⁴⁵Ca Influx test by using CHO cells of the inventive compounds was measured by using Chinese Hamster Ovary (CHO, ATCC, No. CCL-61) cell whose cDNA of VR1 (pUHG102 VR1 plasmid) was transfected, which can control the expression of VR1 according to the presence of tetracycline and Tetracycline on/off system (pTet off regulatory plasmid, Clontech. Inc., USA) that the expression of VR1 is induced by removing tetracycline from the medium.

The CHO cells were poured onto 24 well plates to the extent that its density reaches at 30% and incubated for 24 hours at 37° C. The culture medium was exchanged to tetracycline free medium to induce the expression of VR1 and tested after 36 hours.

In radioactive ⁴⁵Ca uptake experiment, The cells were incubated in 500 ml of DMEM medium (Dulbecco's modified Eagles medium: Gibco-BRL, 31600-083) containing free of serum and 1.8 mM CaCl₂ for 10 minutes at 37*C. Together with 0.25 mg/ml BSA (Sigma A2153, USA), 1 Ci/ml ⁴⁵Ca(5-30 Ci/g used, ICN. Co., 62005 RT, U.S.A.), the test samples with increasing concentrations were added to each well. At the quenching moment of the incubation with ⁴⁵Ca, the cultured cells were removed from the medium, washed three times with cool PBS buffer solution containing 1.8 mM CaCl₂ and 400 μl of RIPA buffer solution (50 mM Tris pH 7.4; 150 mM sodium chloride; 0.1% SDS; 1% sodium deoxycholate), was added in each well to homogenize the cells. The plates were stirred for 20 minutes slowly and 300 μl of cell lysate was transferred to scintillation vials from each wells. The radioactivity was determined by scintillation counter.

The data were assessed by determining four wells per each data point in each experiment and analyzed in computer by being transformed into Hill equation. The experiments were determined in triplicate in each sample comprising inventive compounds and control groups.

In order to determine the antagonistic activity, ⁴⁵Ca²⁺-uptake stimulating-mixture was added with 50 nM capsaicin and the antagonistic activity was determined by the method for the agonistic activity. In case that 10 μM a certain compound cannot change the capsaicin-inducing activity, the compound shall be regarded as an agonist.

The result of the vanilloid receptor affinity and Ca uptake test of each compound was shown in Table 8.

TABLE 8

| Compound | Code | Ki (nM) (VR1/CHO) | | EC₅₀ (nM) (VR1/CHO) | | IC₅₀ (nM) (VR1/CHO) | |
|---|---|---|---|---|---|---|---|
| Capsazepine | | 1350 | (±50) | NE | | 520 | (±12) |
| 28 | JYL-1627 | 1092 | (±145) | NE | | 470.2 | (±197.8) |
| 29 | MY-594 | 926 | (±74) | 2008 | (±198) | NE | |
| 30 | SU-190 | 802 | (±187) | >7062 | | NE | |
| 31 | MY-546 | 1308.3 | (±209.8) | NE | | 579 | (±42.5) |
| 32 | MY-570 | 1328.4 | (±311.1) | NE | | 635 | (±51.8) |
| 33 | SU-308 | 1920.8 | (±333.7) | 12340 | (±2922) | NE | |
| 34 | SU-306 | 2271.6 | (±731.9) | NE | | NE | |
| 35 | SU-66 | 1041.8 | (±72.8) | 1233 | | 212.5 | (±85.3) |
| 36 | MY-650 | 396 | (±62) | 809 | (±126) | NE | |
| 37 | SU-154 | 211.6 | (±39.6) | NE | | 93.67 | (±14) |
| 38 | SU-288 | 623.5 | (±152.3) | 1352 | (±136) | NE | |
| 39 | SU-276 | 220.6 | (±54.5) | NE | | 757.4 | (±65) |
| 40 | SU-552 | 535.6 | (±89.1) | weak | | NE | |
| 41 | SU-530 | 404.8 | (±15.2) | Weak | | Weak | |
| 45 | JYL-1635 | 6375.3 | (±3059) | 3504 | (±1387) | 6589 | (±1986) |

| | | | | | | |
|---|---|---|---|---|---|---|
| 60 | JYL-1371 | 4257 | (±372) | NE | 465 | (±103) |

| 61 | LJO-310 | 481.1 | (±66.9) | Weak | | Weak |
|---|---|---|---|---|---|---|
| 62 | JYL-1453 | 3495 | (±621) | 1055.4 | (±35.4) | NE |
| 63 | JYL-1455 | 5309 | (±725) | 1963 | (±402) | NE |

| | | | | | |
|---|---|---|---|---|---|
| 64 | SU-578 | 545.8 | (±52.7) | Weak | NE |

### EXPERIMENTAL EXAMPLE 3

### Acetic Acid-Induced Writhing Test

The acetic acid-induced writhing test for testing the analgesic activity of inventive compounds prepared from above Examples was performed by the procedure described in the literature (Lee, J. W., Bioorganic & Medicinal Chemistry, pp 1713-1720, 2001).

Male ICR mice having its mean body weight of 25 g (CD-1; Biogenomics Co. Korea) were reared in lighting controlled environment (12 hrs on/12 hrs off) maintaining with temperatures at 22±2° C. and humidity at 50±5% and allowed to eat a diet and to drink tap water ad lib.

Mice were fasted overnight prior to testing and adopted to the environment.

0.3 ml of acetic acid solution (1.2%) was administrated in the mice intraperitoneally and then the mice were put into the transparent acryl box (15×15×15 cm). 5 minutes later, the number of abdominal constrictions was counted for 20 minutes. Each group consisting of ten mice was pretreated with test compounds or solvent (0.2 ml, i.p.) 30 mins before the injection of acetic acid. Test compounds were dissolved in the mixture of ethanol/Tween-80/saline (Oct. 10, 1980) or cremophor EL/DMSO/d-water (Oct. 10, 1980).

Analgesic activity of each drug was determined at several different concentrations.

The index of analgesic activity (eff) was defined as below Empirical formula 1.
Analgesic activity (eff)=100−{(No. of abdominal constriction of test group/No. of abdominal constriction of control group)×100} [Empirical formula 1]

Analgesic activity was expressed as the reduction in the number of abdominal constrictions, of control animals (vehicle-pretreated mice) and animals pretreated with test compounds. ED₅₀, the concentration of the test group to reduce 50% of the number of writhes and the result was shown in Table 9.

TABLE 9

| Thiourea | ED₅₀ (μg/kg) | N-hydroxy thiourea | ED₅₀ (μg/kg) |
|---|---|---|---|
| KJM-429 | 1.410 (±320) | 28 (JYL-1627) | 1.560 (±270) |
| JYL-511 | 0.022 (±0.118) | 29 (MY-594) | 0.103 (±0.061) |
| SC-0030 | 1.257 (±0.0074) | 30 (SU-190) | 1.072 (±0.151) |
| JYL-827 | 2.620 (±2.380) | 35 (SU-66) | 2.600 (±1.100) |
| JYL-1433 | 7.429 (±8.4) | 37 (SU-154) | 0.065 (±0.056) |

Ref. Ketorolac ED₅₀(μg/kg) = 2820

Comparing with the activity of thiourea compound JYL-827 and 1433 disclosed in Korean Patent application No. 2001-50093, the inventive compounds 35 (SU-66) and 37 (SU-154) showed stronger analgesic effect.

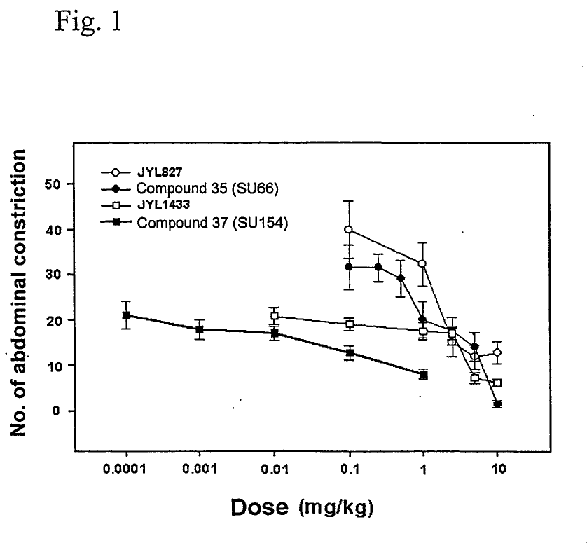

Table 10 shows the order of 37 (SU-154)>JYL-1433, 35 (SU-66)>JYL-827 in analgesic effect. Especially, compound 37 (SU-154) in present invention exhibited 43,000-fold stronger effect than that of Ketorolac, one of the most analgesic compounds in prior art gee Table 10 and FIG. 1).

The test results demonstrated that analgesic effect of the compounds used in this experiment is potent, and in particular, it is significant to clarify that vanilloid receptor antagonist can exhibit such potent analgesic effect, and the result suggests that vanilloid receptor antagonist has potential as an analgesic agent.

TABLE 10

| THIOUREA | N-HYDROXY THIOUREA |
|---|---|
| 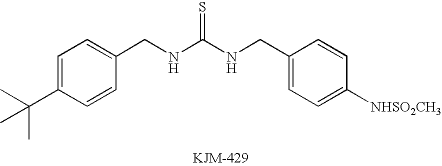 | 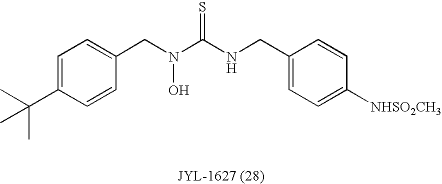 |
| 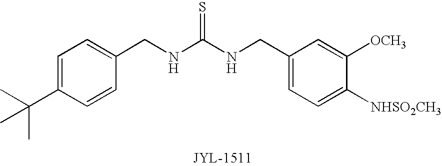 | 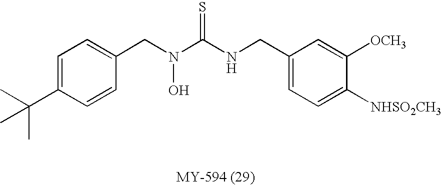 |
| 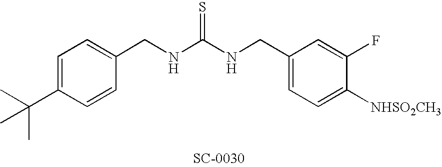 | 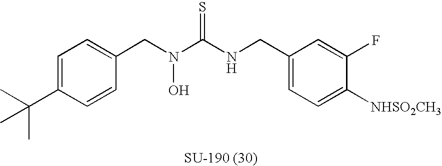 |
| 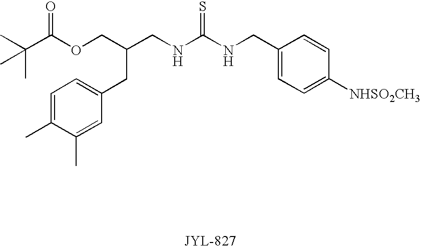 | 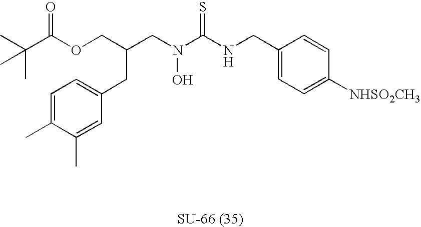 |
| 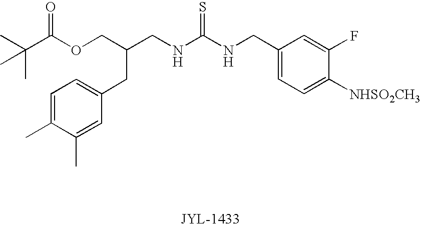 | 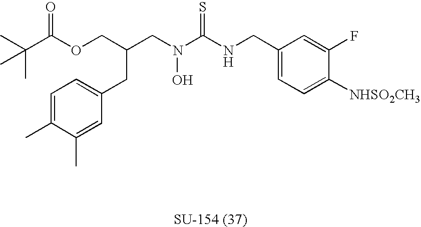 |

### EXPERIMENTAL EXAMPLE 4

### Toxicity Test

The acute toxicity tests on ICR mice (mean body weight 25±5 g) and Sprague-Dawley rats (235±10 g) were performed using the compounds 35 and 37. Each group consisting of 3 mice or rats was administrated intraperitoneally with 20 mg/kg, 10 mg/kg and 1 mg/kg of test compounds or solvents (0.2 ml, i.p.), respectively and observed for 24 hrs.

There were no treatment-related effects on mortality, clinical signs, body weight changes and gross findings in any group or either gender. These results suggested that the compounds prepared in the present invention were potent and safe.

Hereinafter, the formulating methods and kinds of excipients will be described, but the present invention is not limited to them. The representative preparation examples were described as follows.

Preparation of powder

| | | |
|---|---|---|
| Compound 35 | 500 | mg |
| Corn Starch | 100 | mg |
| Lactose | 100 | mg |
| Talc | 10 | mg |

Powder preparation was prepared by mixing above components and filling sealed package.

Preparation of tablet

| | | |
|---|---|---|
| Compound 37 | 100 | mg |
| Corn Starch | 100 | mg |
| Lactose | 100 | mg |
| Magnesium Stearate | 2 | mg |

Tablet preparation was prepared by mixing above components and entabletting.

Preparation of capsule

| | | |
|---|---|---|
| Compound 35 | 50 | mg |
| Lactose | 50 | mg |
| Magnesium Stearate | 1 | mg |

Tablet preparation was prepared by mixing above components and filling gelatin capsule by conventional gelatin preparation method.

Preparation of injection

| | |
|---|---|
| Compound 37 | 100 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of liquid

| | | |
|---|---|---|
| Compound 35 | 1 | g |
| Sugar | 10 | g |

| | |
|---|---|
| Citric acid | 0.05˜0.3% |
| Vitamin C | 0.1˜1% |
| Lemon flavor | optimum amount |
| Distilled water | optimum amount |

Liquid preparation was prepared by dissolving active component, adding lemon flavor and distilled water and then filling all the components in 100 ml brown bottle and sterilizing by conventional liquid preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

The novel N-hydroxy thiourea, urea and amide derivatives compounds and the phamaceutical composition comprising same according to the present invention act as vanilloid receptor-1 antagonists and analgesics so the inventive compounds are useful in the prevention, alleviation or treatment of pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease, inflammatory disease or urgent urinary incontinence, etc.

1. A compound represented by the following general formula (I), the pharmaceutically acceptable salt or the isomer thereof: 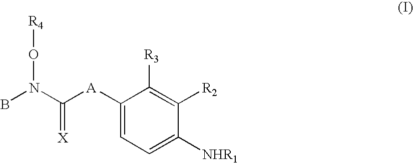
wherein
X is an oxygen or sulfur atom;
A is an aminomethylene or methylene group;
B is a 4-tert-butylbenzyl, a 3,4-dimethylphenylpropyl, an oleyl or 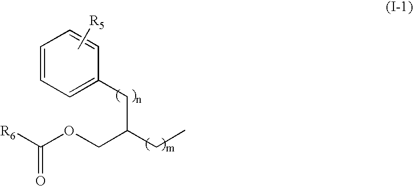
group wherein m is integer of 0 or 1 and n is 1 or 2;
R₁ is a halogen-substituted or unsubstituted lower alkylsulfone having 1 to 5 carbon atoms, arylsulfone or a lower alkylcarbonyl group having 1 to 5 carbon atoms;
R₂ is a hydrogen atom, a methoxy group or halogen atom;
R₃ is a hydrogen atom, a methoxy group or halogen atom;
R₄ is a hydrogen atom or a lower alkyl group having 1 to 5 carbon atoms;
R₅ is a hydrogen atom or a lower alkyl group having 1 to 5 carbon atoms;
R₆ is a lower alkyl group having 1 to 5 carbon atoms or a phenyl group.

2. The compound according to claim 1 represented by the following general formula (I), the pharmaceutically acceptable salt or the isomer thereof: 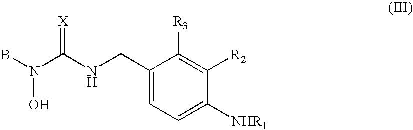
wherein
X is an oxygen atom or a sulfur atom;
R₁ is a halogen-substituted or unsubstituted lower alkylsulfone having 1 to 5 carbon atoms, arylsulfone or a lower alkylcarbonyl group having 1 to 5 carbon atoms;
R₂ is a hydrogen atom, a methoxyl group or a halogen atom;
R₃ is a hydrogen atom or a halogen atom;
B is 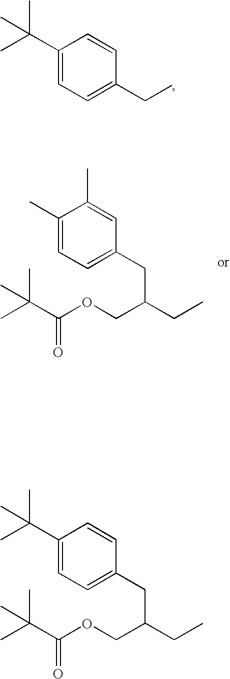
group.

3. The compound according to claim 2 wherein said compound is at least one selected from the group consisting of;
N-(4-tert-butylbenzyl)-N-hydroxy-N-[4-(methylsulfonylamino)benzyl]thiourea,
N-(4-tert-butylbenzyl)-N-hydroxy-N-[3-methoxy-4-(methylsulfonylamino) benzyl]thiourea,
N-(4-tert-butylbenzyl)-N-hydroxy-N-[3-fluoro-4-(methylsulfonylamino)benzyl] thiourea,
N-(4-tert-butylbenzyl)-N-hydroxy-N-[3-chloro-4-(methylsulfonylamino)benzyl] thiourea,
N-(4-tert-butylbenzyl)-N-hydroxy-N-[4-(methylsulfonylamino)-3-nitrobenzyl] thiourea,
N-(4-tert-butylbenzyl)-N-hydroxy-N-[2-fluoro-4-(methylsulfonylamino)benzyl] thiourea,
N-(4-tert-butylbenzyl)-N-hydroxy-N-[2-chloro-4-(methylsulfonylamino)benzyl] thiourea,
N-[2-(3,4-dimethylbenzyl)-3-(pivaloyloxy)propyl]-N-hydroxy-N-[4-(methylsulfonyl amino)benzyl] thiourea,
N-[2-(3,4-dimethylbenzyl)-3-(pivaloyloxy)propyl]-N-hydroxy-N-[3-methoxy-4-(methylsulfonylamino)benzyl] thiourea,
N-[2-(3,4-dimethylbenzyl)-3-(pivaloyloxy)propyl]-N-hydroxy-N-[3-fluoro-4-(methylsulfonylamino)benzyl] thiourea,
N-[2-(3,4-dimethylbenzyl)-3-(pivaloyloxy)propyl]-N-hydroxy-N-[2-fluoro-4-(methylsulfonylamino)benzyl] thiourea,
N-[2-(3,4-dimethylbenzyl)-3-(pivaloyloxy)propyl]-N-hydroxy-N-[2-chloro-4-(methylsulfonylamino)benzyl] thiourea,
N-[2-(4-tert-butylbenzyl)-3-pivaloyloxy)propyl]-N-hydroxy-N-[4-(methylsulfonyl amino)benzyl] thiourea, and
N-[2-(4-tert-butylbenzyl)-3-(pivaloyloxy)propyl]-N-hydroxy-N-[3-fluoro-4-(methylsulfonylamino)benzyl] thiourea.

4. The compound according to claim 1 represented by the following general formula (IV), the pharmaceutically acceptable salt or the isomer thereof: 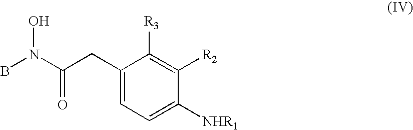
wherein
R₁ is a halogen-substituted or unsubstituted lower alkylsulfone having 1 to 5 carbon atoms, arylsulfone or a lower alkylcarbonyl group having 1 to 5 carbon atoms;
R₂ is a hydrogen atom, a methoxyl group or a halogen atom;
R₃ is a hydrogen atom or a halogen atom;
B is 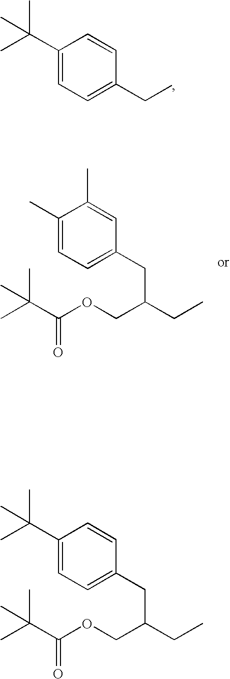
group.

5. The compound according to claim 4 wherein said compound is N-(4-tert-butylbenzyl)-N-hydroxy-[4-(methylsulfonylamino)phenyl] acetamide.

6. A compound represented by general formula (II), the pharmaceutically acceptable salt or the isomer thereof: 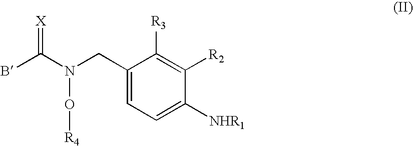
wherein
X is an oxygen or sulfur atom;
B′ is B or a secondary amine substituted with B,
wherein B is a 4-tert-butylbenzyl, a 3,4-dimethylphenylpropyl, an oleyl or 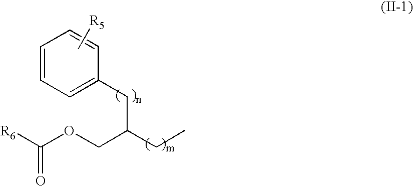
group, wherein m is integer of 0 or 1 and n is 1 or 2;
R₁ is a halogen-substituted or unsubstituted lower alkylsulfone having 1 to 5 carbon atoms, arylsulfonyl group or lower alkylcarbonyl group having 1 to 5 carbon atoms;
R₂ is a hydrogen atom, a methoxy group or halogen atom;
R₃ is a hydrogen atom, a methoxy group or halogen atom;
R₄ is a hydrogen atom or a lower alkyl group having 1 to 5 carbon atoms;
R₅ is a hydrogen atom or a lower alkyl group having 1 to 5 carbon atoms;
R₆ is a lower alkyl group having 1 to 5 carbon atoms or a phenyl group.

7. The compound according to claim 6 represented by general formula (V), the pharmaceutically acceptable salt or the isomer thereof: 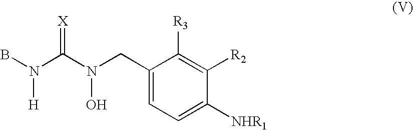
wherein
X is an oxygen atom or a sulfur atom;
R₁ is a halogen-substituted or unsubstituted lower alkylsulfone having 1 to 5 carbon atoms, arylsulfonyl group or lower alkylcarbonyl group having 1 to 5 carbon atoms;
R₂ is a hydrogen atom or a halogen atom;
R₃ is a hydrogen atom;
B is 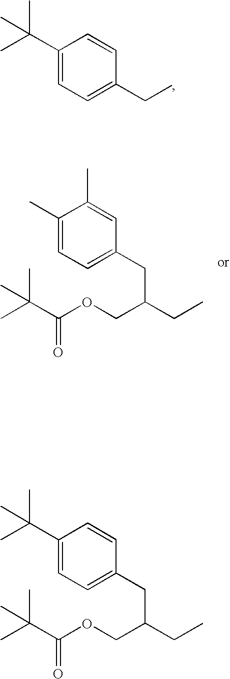
group.

8. The compound according to claim 7 wherein said compound is at least one selected from the group consisting of;
N-(4-tert-butylbenzyl)-N-hydroxy-N-[4-(methylsulfonylamino)benzyl]thiourea,
N-[2-(3,4-dimethylbenzyl)-3(pivaloyloxy)propyl]-N-hydroxy-N-[4-(methylsulfonyl amino)benzyl]thiourea,
N-(4-tert-butylbenzyl)-N-hydroxy-N-[4-(methylsulfonylamino)benzyl]urea,
N-[2-(3,4-dimethylbenzyl)-3(pivaloyloxy)propyl]-N-hydroxy-N-[3-fluoro-4-(methylsulfonylamino)benzyl]thiourea.

9. The compound according to claim 6 represented by general formula (VI), the pharmaceutically acceptable salt or the isomer thereof: 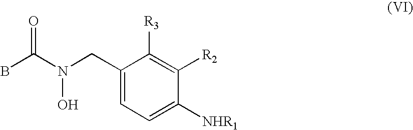
wherein
R₁ is a halogen-substituted or unsubstituted lower alkylsulfone having 1 to 5 carbon atoms, arylsulfonyl group or lower alkylcarbonyl group having 1 to 5 carbon atoms;
R₂ is a hydrogen atom, a methoxyl group or a halogen atom;
R₃ is a hydrogen atom, a methoxyl group or a halogen atom;
B is 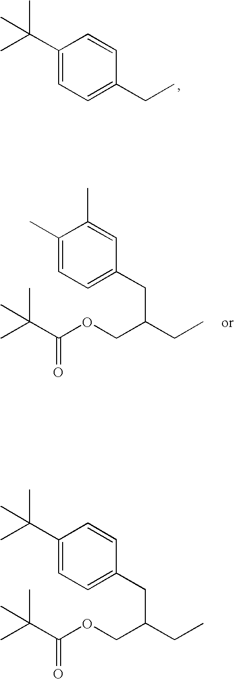
group.

10. The compound according to claim 9 wherein said compound is N-hydroxy-N-[4 (methylsulfonylamino)benzyl]-2-(4-tert-butylphenyl)acetamide.

11. A pharmaceutical composition comprising the compound of general formula (I) as set forth in claim 1 as an active ingredient in amount effective amount for an antagonist of vanilloid receptor together with pharmaceutically acceptable carriers or diluents.

12. A pharmaceutical composition comprising the compound of general formula (I) as set forth in claim 1 as an active ingredient in amount effective to alleviate or treat pain diseases or inflammatory diseases together with pharmaceutically acceptable carriers, excipients or diluents.

13. A pharmaceutical composition comprising an efficient amount of the compound represented by general formula (II) as set forth in claim 6 as an active ingredient in amount effective for an antagonist of vanilloid receptor together with pharmaceutically acceptable carriers or diluents.

14. A pharmaceutical composition comprising the compound of general formula (II) as set forth in claim 6 as an active ingredient in amount effective amount to alleviate or treat pain disease together with pharmaceutically acceptable carriers or diluents.

15. The pharmaceutical composition according to claim 12 or 14 wherein said pain disease is at least one selected from the group consisting of pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, a respiratory disorder such as asthma or chronic obstructive pulmonary disease, irritation of skin, eye or mucous membrane, fervescence, stomach-duodenal ulcer, inflammatory bowel disease caused by the vanilloid receptor antagonistic activity.

16. A pharmaceutical composition comprising the compound of any one of claims 1 to 10 as an active ingredient in amount effective for analgesic and anti-inflammation together with pharmaceutically acceptable carriers or diluents.

17. A pharmaceutical composition comprising the compound of any one of claims 1 to 10 as an active ingredient together with pharmaceutically acceptable carriers or diluents for preventing and treating urgent urinary incontinence.

18. Use of the compound of any one of claim 1 to 10 for the preparation of therapeutic agent for the preventing and treating pain disease or inflammatory disease by showing vanilloid receptor-antagonistic activity in human or mammal.
